(19) United States
(12) Patent Application Publication
(10) Pub. No.: US 2021/0308211 A1
(43) Pub. Date: Oct. 7, 2021

(54) METHOD OF TREATING VIRAL INFECTIONS

(71) Applicants: IMMUPHARMA FRANCE SA, Mulhouse (FR); CENTRE NATIONAL DE LA RECHERCHE SCIENTIFIQUE (CNRS), Paris (FR)

(72) Inventors: Robert H. Zimmer, Mulhouse (FR); Sylviane Muller, Strasbourg (FR)

(21) Appl. No.: 17/215,089

(22) Filed: Mar. 29, 2021

Related U.S. Application Data
(60) Provisional application No. 63/001,423, filed on Mar. 29, 2020.

(51) Int. Cl. A61K 38/16; A61K 38/10

(52) U.S. Cl. CPC A61K 38/16; A61K 38/10

(57) ABSTRACT

The present disclosure relates to methods of treating, preventing, or ameliorating at least one symptom of a viral infection or virus-induced immunopathology in a subject in need thereof, as well as methods of modulating the immune response in a subject having a viral infection. The methods include: administering an effective amount of a pharmaceutical composition that includes an effective amount of a peptide having the amino acid sequence as set forth in SEQ ID NO: 4, SEQ ID NO: 5, SEQ ID NO: 6, SEQ ID NO: 7, a salt form thereof, or combination thereof; and at least one pharmaceutically acceptable carrier or excipient.

### CROSS-REFERENCE TO RELATED APPLICATION

This application claims priority to and the benefit of U.S. Provisional Application No. 63/001,423, filed 29 Mar. 2020, titled METHOD OF TREATING VIRAL INFECTIONS, which incorporated herein in its entirety for all purposes.

### INCORPORATION BY REFERENCE

An electronic version of the Sequence Listing file name: IMM0043US2_Sequenc_Listing_ST25_26MAR2021.txt, size: 7.15 KB, created 26 Mar. 2021 using Patent-In 3.5, and Checker 4.4.0, containing SEQ ID NOs: 1-8 is filed herewith and is hereby incorporated by reference in its entirety.

### BACKGROUND

### 1. Field of the Discovery

The present disclosure relates to modified peptides, and their use for treating virus-induced immunopathology, including virus-induced pneumopathy or that observed in viral pneumonia, including Coronavirus induced pneumopathy, e.g., that observed in Middle East Respiratory Syndrome (MERS), Severe Acute Respiratory Syndrome (SARS), and Coronavirus Disease 2019 (COVID-19).

### 2. Background Information

While a virus can directly cause tissue damage during a viral infection (e.g., a lytic viral infection), so too can the host immune system. The host immune response can mediate disease and in particular, excessive inflammation. The stimulation of the innate immune system and adaptive immune system in response to viral infections destroys infected cells, which may lead to severe pathological consequences to the host. The damage caused by the immune system is known as virus-induced immunopathology.

Virus-induced immunopathology can be caused by, e.g., the excessive release of antibodies, interferons and pro-inflammatory cytokines, activation of the complement system, or hyperactivity of cytotoxic T cells. Secretion of interferons and other cytokines can trigger cell damage, fever, muscle aches, fatigue, cough, etc. For example, in severe cases of certain viral infections, as in the avian H5N1 influenza pandemic in 2005, aberrant induction of the host immune response can elicit a cytokine storm, causing severe virus-induced immunopathology. One example of virus-induced immunopathology is virus-induced pneumopathy or the pathology observed during viral pneumonia.

Viral pneumonia is a disease where there is a viral causation of oxygen and carbon dioxide gas exchange abnormalities at the level of the alveoli, which is secondary to viral-mediated and/or immune response-mediated inflammation. The traditional role of viral pneumonia was as a disease found predominantly in the very young, the elderly, and those exposed to influenza. In the past, the diagnosis of viral pneumonia was predicated on it being somewhat a diagnosis of exclusion. Once bacterial pneumonia has been excluded, then viral pneumonia diagnosis was entertained.

During viral pneumonia, the submucosa of the alveoli is targeted, causing inflammation and secondary edema, microhemorrhage, and cellular immune reaction. The cellular reaction consists of mononuclear lymphocytes and progresses to the recruitment of polymorphonuclear leukocytes (PMNs), which play a central role in inflammation and can be the cause of significant tissue damage. Furthermore, CD4 and CD8 cells are involved, beginning a cascade of immune product secretion that can increase vascular permeability, thereby resulting in edema. Ultimately, this can lead to interstitial pneumonia, pulmonary edema, and cardiogenic shock, which can ultimately result in death.

SARS is a contagious and sometimes fatal respiratory illness caused by the Severe Acute Respiratory Syndrome Coronavirus (SARS-CoV) that appeared in 2002 in China. It spread worldwide within a few months, though it was quickly contained. SARS-CoV is transmitted through droplets that enter the air when someone with the disease coughs, sneezes, or talks. No known transmission has occurred since 2004. SARS symptoms include fever, dry cough, headache, muscle aches, and difficulty breathing from viral pneumonia. No treatment exists for SARS except supportive care.

MERS is a viral respiratory disease caused by the Middle East Respiratory Syndrome Coronavirus (MERS-CoV) that was first identified in Saudi Arabia in 2012. MERS typically presented with a fever, cough and shortness of breath, commonly associated with pneumonia. Approximately 35% of patients confirmed to have MERS have died. No vaccine or treatment exists for MERS, except supportive care.

COVID-19 is a viral respiratory disease caused by Severe Acute Respiratory Syndrome Coronavirus 2 (SARS-CoV-2) that was first identified in china in 2019. SARS-CoV-2 since its identification has spread globally, and part of an ongoing pandemic with over 700,000 confirmed cases to date. Common symptoms include fever, cough, muscle pain, sore throat, sputum production, and shortness of breath, as a result of pneumonia. The clinical spectrum of COVID-19 ranges from mild to critically ill cases. While the mortality rate of COVID-19 is not entirely clear, it is clear that a major driver of its mortality is the severe pneumonia SARS-CoV-2 causes.

Viral pneumonia treatment generally revolves around supportive care—e.g., supplemental oxygen, airway augmentation, monitoring and replacement of fluid deficits, symptomatic control of temperature and cough, reduce oxygen demand through rest, and treatment of comorbidities and/or concomitant bacterial pneumonia. Thus, there is a lack of safe, tolerable, and effective treatments for viral pneumonia.

Thus, there exists in the art an ongoing need for therapeutic interventions to treat virus-induced immunopathology, such as virus induced pneumopathy and viral pneumonia, such as that observed in COVID-19. In particular, there exists a need for therapeutic interventions that target key cellular processes involved in the initiation and persistence of inflammation in viral infections, such as viral pneumonia, a significant cause of virus-induced pneumopathy. Accordingly, there is a need to provide therapeutic interventions capable of treating, preventing and/or ameliorating the symptoms of virus-induced immunopathology.

### SUMMARY

The present description provides peptides and compositions having the same for surprising and unexpected use in methods to prevent, treat, and/or ameliorate at least one symptom of a viral infection or virus-induced immunopathology (such as virus-induced pneumopathy or viral pneumonia). The chemically modified peptides as described herein are derived from the U1-70K spliceosomal protein. The described peptides and compositions comprising effective amounts of the same are effective for treating, preventing and/or ameliorating the symptoms of a viral infection or virus-induced immunopathology (such as virus-induced pneumopathy or viral pneumonia). Accordingly, in certain additional aspects, the disclosure provides methods of making and using the described peptides and compositions comprising the same for the treatment, prevention and/or amelioration of the symptoms of a viral infection or virus-induced immunopathology (such as a viral induced pneumopathy or viral pneumonia), such as reducing viral induced inflammation (e.g., virus-induced inflammation in the lung).

Thus, in one aspect the present description provides chemically modified peptides of SEQ ID NOs: 1, 2, 4, 5, 6, and 7, including derivatives, analogs and salt forms thereof.

In any aspect or embodiment described herein, the description provides an isolated peptide comprising or consisting of the amino acid sequence of SEQ ID NO: 1:

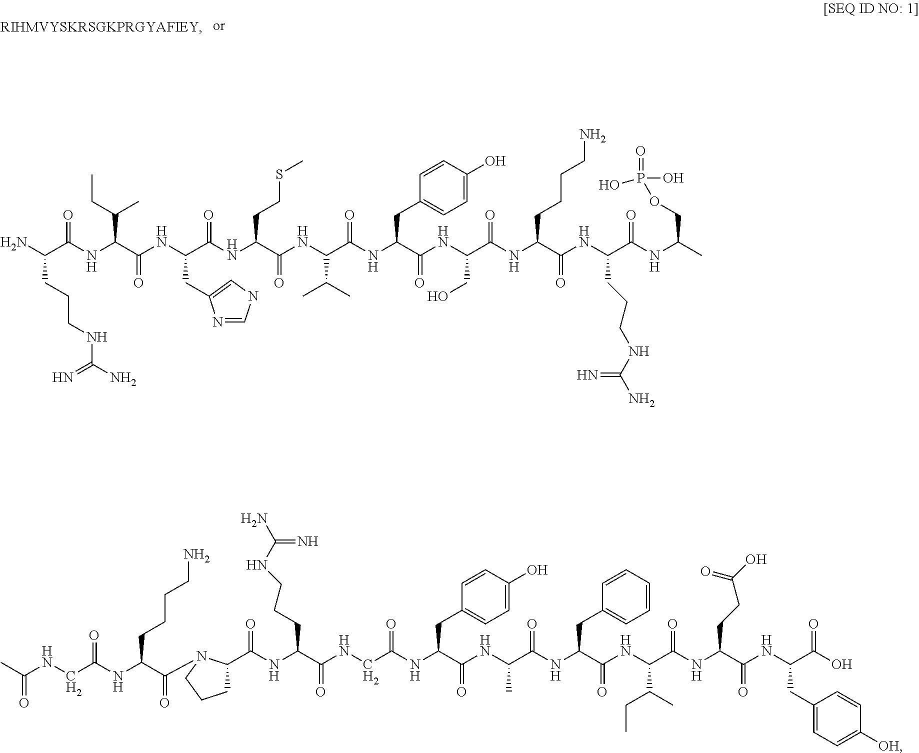

or salt thereof, having at least one post-translational modification selected from the group consisting of phosphorylation of a serine residue, oxidation of a methionine residue, and acetylation of a lysine residue, and combinations thereof. In an embodiment of this aspect, the description provides a composition comprising an isolated and/or chemically modified peptide (recombinant or synthesized) having or consisting of the amino acid sequence of SEQ ID NO: 1, or salt thereof, wherein the peptide comprises a phosphoserine at position 10 (i.e., “P140 peptides” or SEQ ID NO: 4). In any aspect or embodiment described herein, the description provides an isolated and/or chemically modified peptide (recombinant or synthesized) having or consisting of the amino acid sequence of SEQ ID NO: 1, or salt thereof, wherein the peptide comprises a phosphoserine at position 10, and an oxidized Methionine residue at position 4 [SEQ ID NO: 6].

In any aspect or embodiment described herein, the peptide of SEQ ID NO:1 also comprises an acetylated lysine residue. In particular, said peptide of SEQ ID NO: 1 comprises a phosphoserine at position 10, and an oxidized Methionine residue at position 4, and an acetylation of one or both of the lysine at position 8 and 12, and more particularly further comprises a phosphoserine at position 7.

In any aspect or embodiment described herein, the description provides an isolated and/or chemically modified peptide (recombinant or synthesized), or a salt thereof, comprising or consisting of the amino acid sequence:

[SEQ ID NO: 2]
IHMVYSKRSGKPRGYAFIEY,

in which the Serine (S) at position 9 is phosphorylated, and the Methionine (M) at position 3 is oxidized SEQ ID NO: 7].

In any aspect or embodiment described herein, the description provides a peptide of compound I having the following formula:

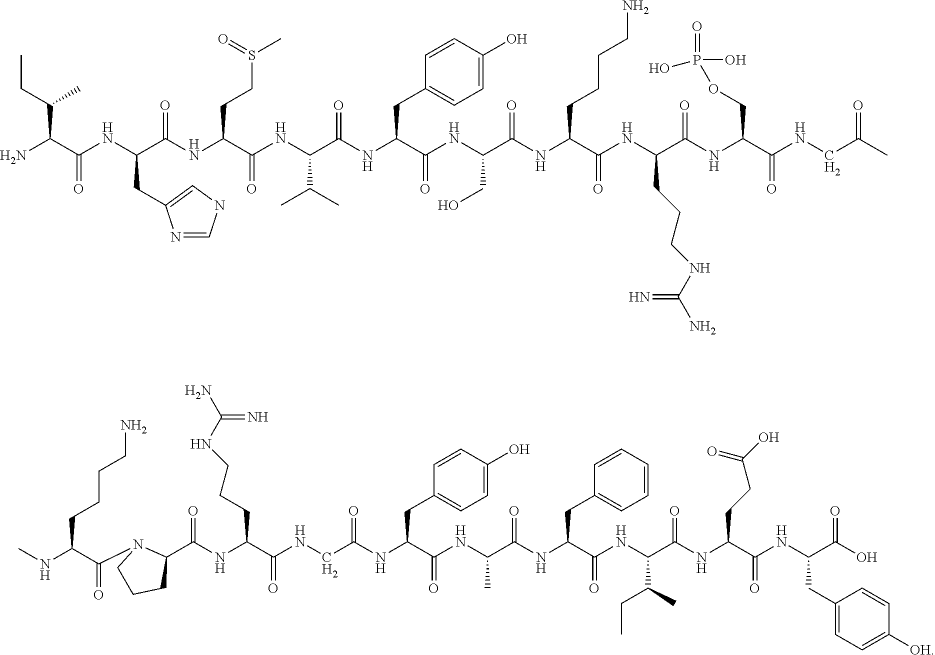

Compound I can also be represented by:

[SEQ ID NO: 7]
IHM(O)VYSKRS(PO₃H₂)GKPRGYAFIEY

in which “M(O)” represents oxidized methionine, and “S(PO₃H₂)” represents phosphoserine.

These peptides are derived from the human U1 snRNP 70 kDa protein (SEQ ID NO: 3), and correspond to the region delimited by the amino acid segment extending from the residue 132 to the residue 151 of SEQ ID NO: 3. Formally, the residue which is phosphorylated corresponds to the amino acid at the position 140 from the first methionine of SEQ ID NO: 3, and the residue which is oxidized corresponds to the amino acid at the position 134 from the first methionine of SEQ ID NO: 3.

In additional aspects, the description provides an isolated and/or chemically modified peptide (recombinant or synthesized) comprising or consisting of the amino acid sequence of SEQ ID NO: 2, or salt thereof, having at least one post-translational modification selected from the group consisting of phosphorylation of a serine residue, oxidation of a methionine residue, and acetylation of a lysine residue, and combinations thereof. In an embodiment of this aspect, the description provides a composition comprising an isolated and/or chemically modified peptide (recombinant or synthesized) having or consisting of the amino acid sequence of SEQ ID NO: 2, or salt thereof, wherein the peptide comprises a phosphoserine at position 9, and an oxidized Methionine residue at position 3. In any aspect or embodiment described herein, the peptide of SEQ ID NO:2 further comprises an acetylated lysine residue.

In certain embodiments, the description provides a peptide of compound II having the following formula:

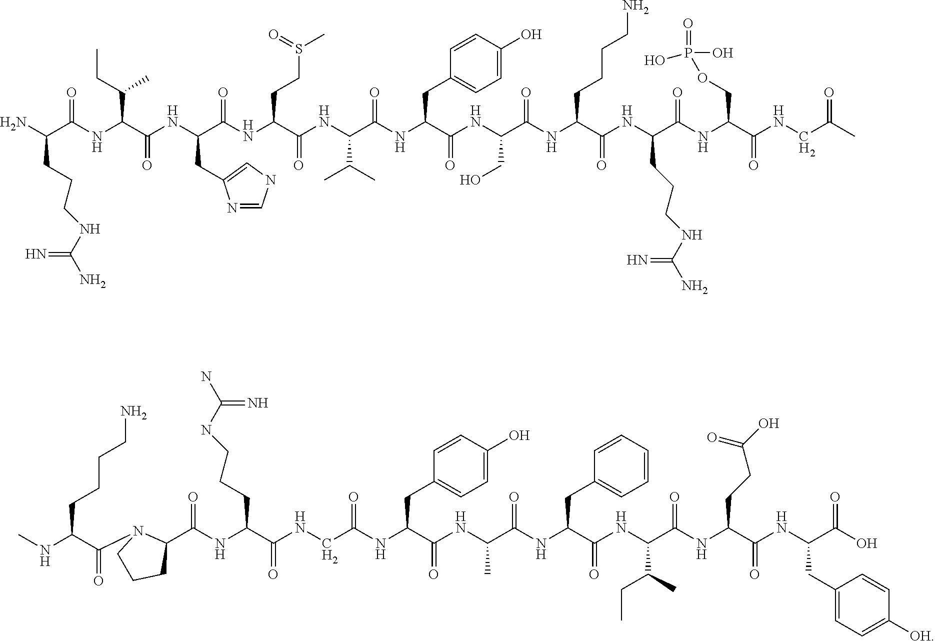

Compound II can also be represented by:

[SEQ ID NO: 6]
RIHM(O)VYSKRS(PO₃H₂)GKPRGYAFIEY

in which M(O) represents oxidation of methionine, and S(PO₃H₂) represents the phosphorylation of serine.

Thus, the description provides peptides, or a salt thereof, comprising or consisting of the amino acid sequence chosen among the group consisting of SEQ ID NO: 4, SEQ ID NO: 5, SEQ ID NO: 6, and SEQ ID NO: 7, and compositions including one or more of the peptides.

In an additional embodiment, the description provides a pharmaceutical composition comprising, consisting essentially of, or consisting of an effective amount of at least one peptide, or salt thereof, selected from the group consisting of: the amino acid sequence SEQ ID NO: 2, comprising a phosphoserine at position 9, and oxidized Methionine at position 3 [SEQ ID NO: 7]; the amino acid sequence SEQ ID NO: 1, comprising a phosphoserine at position 10, and an oxidized Methionine at position 4 [SEQ ID NO: 6]; the amino acid sequence of SEQ ID NO: 1, or salt thereof, comprising a phosphoserine at position 10 [SEQ ID NO: 4 or P140]; the amino acid sequence of SEQ ID NO: 2, or salt thereof, comprising a phosphoserine at position 9 [SEQ ID NO: 7]; and a combination thereof.

In another aspect the present description provides pharmaceutical compositions comprising an effective amount of one or more of the peptides as described herein, and an effective amount of an excipient or carrier.

A further aspect of the present disclosure provides a pharmaceutical composition comprising: one or more peptides of the present disclosure; and a pharmaceutically acceptable carrier or excipient.

An additional aspect of the present disclosure provides a pharmaceutical composition comprising, consisting essentially of, or consisting of: an effective amount of a peptide having the amino acid sequence as set forth in SEQ ID NO: 6, SEQ ID NO: 7, salt form thereof, or a combination thereof; and a pharmaceutically acceptable carrier or excipient.

In an additional aspect, the present description provides methods for treating, preventing or ameliorating at least one symptom of a viral infection or virus-induced immunopathology (e.g., virus-induced pneumopathy or viral pneumonia) in a subject in need thereof, the method comprising: providing a subject in need thereof; and administering an effective amount of one or more peptides of the present disclosure or a pharmaceutical composition of the present disclosure, wherein the peptide effectuates the treatment or amelioration of at least one symptom of the viral infection or the virus-induced immunopathology (e.g., virus-induced pneumopathy or viral pneumonia).

In an additional aspect, the present description provides methods for modulating the immune response (such as the immune response in the lung(s)) in a subject having viral infection (e.g., viral pneumonia), the method comprising: providing a subject in need thereof; and administering an effective amount of one or more peptides of the present disclosure or a pharmaceutical composition of the present disclosure, wherein modulating the immune response is effective to treat, prevent, or ameliorate at least one symptom of viral infection (e.g., at least one symptom of virus-induced pneumopathy or viral pneumonia).

In any aspect or embodiment described herein, the viral infection is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof.

In any aspect or embodiment described herein, the virus-induced immunopathology, the virus-induced pneumopathy, or the viral pneumonia is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof.

In any aspect or embodiment described herein, the method results in a decrease of inflammation (e.g., a decrease in lung inflammation), a decrease in edema, a decrease in tissue damage (e.g., tissue damage in the lung), ameliorates at least one symptom of the viral infection (e.g., at least one symptom of viral pneumonia), ameliorates at least one symptom of virus-induced immunopathology (e.g., at least one symptom of a virus pneumopathy or viral induced pneumonia), or a combination thereof.

In any aspect or embodiment described herein, the method treats, prevents, or ameliorates at least one symptom of COVID-19.

In any aspect or embodiment described herein, the method treats, prevents, or ameliorates COVID-19 pathology.

In any aspect or embodiment described herein, the pharmaceutical compositions of the present disclosure may further include at least one additional bioactive agent, e.g., an immunomodulatory agent, e.g., a steroid, anti-malarial, methotrexate or a combination thereof. In any aspect or embodiment described herein, the composition further comprises an effective amount of an excipient or carrier as described herein.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the present disclosure may be utilized in numerous combinations, all of which are expressly contemplated by the present description. These additional advantages objects and embodiments are expressly included within the scope of the present disclosure. The publications and other materials used herein to illuminate the background of the present disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure and, together with the description, serve to explain the principles of the present disclosure. The drawings are only for the purpose of illustrating an embodiment of the present disclosure and are not to be construed as limiting the present disclosure.

FIG. 1. Demonstrates the stability at 37° C. of Compound II [SEQ ID NO: 6] as compared to the stability of the peptide consisting of SEQ ID NO: 4. The graph represents the percentage of stability over the time (expressed in days). Curves A-C represent the stability of Compound II at a concentration of 200, 100 and 50 μg/ml, respectively. Curves D-F represent the stability of the peptide consisting of SEQ ID NO: 1, in which serine at position 10 is phosphorylated at a concentration of 200, 100 and 50 μg/ml, respectively.

FIG. 2. Kaplan-Meier graph representing the cumulative survival rate (in percent) over the time (expressed in weeks) of mice injected with NaCl (line with circles), the peptide consisting of SEQ ID NO: 4 (line with squares) and compound II [SEQ ID NO: 6] according to the present disclosure (lines with triangles).

FIG. 3. Proteinuria score over the time (expressed in weeks) of mice injected with NaCl (line with circles), the peptide consisting of SEQ ID NO: 4 (line with squares) and compound II [SEQ ID NO: 6] according to the present disclosure (lines with triangles).

FIG. 4. Measure of the hypercellularity of MRL/lpr mice cells. Y-axis represents the number of cells/mL of blood (×10⁶), in mice treated with NaCl (circles), the peptide consisting of SEQ ID NO: 1, in which serine at position 10 is phosphorylated (squares) and compound II according to the present disclosure (triangles).

FIG. 5. Measure of the affinity for the HSC70 protein of the peptide consisting of SEQ ID NO: 4. Curves corresponds to the Biacore response over the time (expressed in seconds) by using the peptide consisting of SEQ ID NO: 4 at a concentration of 25 μM(A), 12.5 μM(B), 6.25 μM(C), 3.12 μM(D) and 1.56 μM (E).

FIG. 6. Measure of the affinity of the compound II [SEQ ID NO: 6], for the HSC70 protein. Curves correspond to the Biacore response over the time (expressed in seconds) by using the Compound II at a concentration of 25 μM(A), 12.5 μM(B), 6.25 μM(C), 3.12 μM(D) and 1.56 μM (E).

FIG. 7. CD4⁺ T splenocytes proliferation in the presence of 100 μg CII/mL in the cultures.

FIG. 8. Cellular uptake of fluorescent P140 peptide in 5.4% mannitol or 10% trehalose in MRL/lpr B cells and Raji cells as visualized by flow cytometry. B cells were from 12-14 week-old MRL/lpr mice (primary cells); Raji cells are an established cell line derived in 1963 from B-lymphocyte of a patient with Burkitt's lymphoma. Much less cellular uptake of P140 in both MRL/lpr B cells and Raji cells when the peptide is diluted in trehalose than in mannitol.

FIG. 9. Confocal images of B cells of FIG. 10. All confocal images were taken in the same microscopic settings. Rab9 (red) identifies the late endosomal compartment where P140 localizes before homing into lysosomes DAPI (blue) identifies DNA. The results confirm the flow cytometry results that when in trehalose, P140 peptide (in green) enters B cells much less.

FIG. 10. The anti-inflammatory effect of a P140 phosphopeptide was evaluated when administered locally (intranasally) or systemically (intravenously) in a 15-day model of hypereosinophilic airway inflammation in mice. Briefly, nine-week-old male Balb/c mice were sensitized by intraperitoneal (i.p.) injections of a mixture containing 50 μg OVA and 2 mg alum in 0.1 ml saline. Mice were challenged by i.n. administration of 25 μl of OVA on day 5, then 25 μl of OVA and/or saline on day 12, 13 and 14. Mice were treated by i.v. injection (2 ml/kg) or i.n. administration (1 ml/kg) of P140 or solvent on day 9.

FIGS. 11A, 11B, 11C, 11D, and 11E. Effect of the P140 phosphopeptide on airway inflammatory cell recruitment in an ovalbumin-induced airway hypereosinophilia model in Balb/c mice. Balb/c mice were immunised to OVA (day 0, 1 and 2) and challenged with OVA (day 5) and OVA or saline (day 12, 13 and 14). P140 was administered i.n. (P140-IN) or i.v. (P140-IV) at the dose of 4 mg/kg on day 9. Absolute numbers of (A) eosinophils, (B) neutrophils, (C) macrophages, (D) T cells, and (E) B cells in BAL are shown. Blocks are means and bars are SEM values (n=1 or 6 per group). ###p≤0.001 vs control group and *p≤0.05, **p≤0.01 and ***p≤0.001 vs OVA group.

FIG. 12. Nine-week-old male Balb/c mice were sensitized by intranasal (i.n.) administration of HDM extract (Stallergenes): 1 μg in 25 μl saline on days 0, 1, 2, 3, 4, and 10 μg on days 14 and 21. Mice were challenged by i.n. administration of HDM (1 μg) and/or saline on days 28, 29 and 30. Mice were treated by i.v. injection (2 ml/kg) of P140 or solvent on day 25

FIGS. 13A, 13B, and 13C. Effect of the P140 phosphopeptide on airway reactivity in an HDM-induced asthma model in Balb/c mice. Airway resistance R expressed as cm H₂0·s·mL⁻¹ (A), elastance E expressed as cm H₂0·mL⁻¹ (B) and compliance C expressed as mL·cm H₂⁻¹ (C) at baseline and in response to aerosolized PBS and MCh (50 mg/mL) was assessed with Flexivent®. Blocks are means and bars are SEM values (n=5 to 8 per group). ###p≤0.001 between PBS and MCh nebulisation, and *p≤0.05 between P140 and solvent groups in chronic asthma.

FIG. 14. Effect of the P140 phosphopeptide on airway inflammatory cell recruitment in an HDM-induced asthma model in Balb/c mice. Balb/c mice were sensitized by intranasal (i.n.) administration of HDM (Stallergenes): 1 μg in 25 μl PBS on day 0, 1, 2, 3, 4, and 10 μg on day 14 and 21. Mice were challenged by i.n. administration of HDM and/or PBS on day 28, 29 and 30. Mice were treated by i.v. injection (2 ml/kg) of P140 at the dose of 4 mg/kg or solvent on day 25. Absolute numbers of eosinophils, neutrophils, T cells, B cells, macrophages and DCs in BAL are shown. Blocks are means and bars are SEM values (n=5 to 8 per group). #p≤0.05 and ###p≤0.001 between solvent group in chronic asthma and allergen challenge, and *p≤0.05 between P140 and solvent groups in chronic asthma.

FIGS. 15A and 15B. Body weight (A) and clinical course (B) of CIDP rats treated with P140 peptide (●) compared to untreated rats (□). Injection of P140 peptide is represented by red arrows. Mean values and SEM are indicated.

FIG. 16. Evaluation of lymphocyte subpopulations in isolated salivary glands.

FIG. 17. Evaluation of the level of inflammation in isolated salivary glands.

FIG. 18. Evaluation of the number of FS is isolated salivary glands.

FIG. 19. Effect of the P140 peptide in the murine model of rheumatoid arthritis.

FIG. 20. Effect of the P140 peptide in the murine model of rheumatoid arthritis.

FIG. 21. Effect of the P140 peptide in the murine model of rheumatoid arthritis.

FIG. 22. Effect of the P140 peptide in the murine model of rheumatoid arthritis.

FIG. 23. Effect of the P140 peptide in the murine model of rheumatoid arthritis.

FIG. 24. Effect of the P140 peptide in the murine model of rheumatoid arthritis.

FIG. 25. Effect of the P140 peptide in the murine model of rheumatoid arthritis.

FIG. 26. Effect of the P140 peptide in the murine model of rheumatoid arthritis.

FIG. 27. Effect of the P140 peptide in the murine model of rheumatoid arthritis.

FIG. 28. Effect of the P140 peptide in the murine model of rheumatoid arthritis.

FIG. 29. Effect of the P140 peptide in the murine model of rheumatoid arthritis.

FIG. 30. Effect of the P140 peptide in the murine model of rheumatoid arthritis.

FIG. 31. Evolution of the size of the front straight panes daily, P140 vs NaCl (unpaired T test)

FIG. 32. Evolution of the size of the left front legs daily, P140 vs NaCl (unpaired T test)

FIG. 33. Evolution of inflammation score overnight, P140/NaCl vs Lupuzor™.

### DETAILED DESCRIPTION

The following is a detailed description of the present disclosure provided to aid those skilled in the art in practicing the present disclosure. Those of ordinary skill in the art may make modifications and variations in the embodiments described herein without departing from the spirit or scope of the present disclosure. Although any methods and materials similar or equivalent to those described herein can also be used in the practice or testing of the present disclosure, the preferred methods and materials are now described. Unless otherwise defined, all technical and scientific terms used herein have the same meaning as commonly understood by one of ordinary skill in the art to which this disclosure belongs. The terminology used in the description of the present disclosure herein is for describing particular embodiments only and is not intended to be limiting of the present disclosure. All publications, patent applications, patents, figures and other references mentioned herein are expressly incorporated by reference in their entirety.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the present disclosure.

Where a range of values is provided, it is understood that each intervening value, to the tenth of the unit of the lower limit unless the context clearly dictates otherwise, between the upper and lower limit of that range and any other stated or intervening value in that stated range is encompassed within the present disclosure. The upper and lower limits of these smaller ranges which may independently be included in the smaller ranges is also encompassed within the present disclosure, subject to any specifically excluded limit in the stated range. Where the stated range includes one or both of the limits, ranges excluding either both of those included limits are also included in the present disclosure.

As used herein, the following terms may have meanings ascribed to them below, unless specified otherwise. However, it should be understood that other meanings that are known or understood by those having ordinary skill in the art to which the present disclosure belongs are also possible, and within the scope of the present disclosure.

It must be noted that as used herein and in the appended claims, the singular forms “a”, “and”, and “the” include plural references (i.e., refer to one or to more than one or at least one) to the grammatical object of the article. By way of example, “an element” means one element or more than one element.

The term “about” as it is used herein, in association with numeric values or ranges, reflects the fact that there is a certain level of variation that is recognized and tolerated in the art due to practical and/or theoretical limitations. For example, minor variation is tolerated due to inherent variances in the manner in which certain devices operate and/or measurements are taken. In accordance with the above, the phrase “about” is normally used to encompass values within the standard deviation or standard error.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are coadministered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other steroisomers (diastereomers) thereof, as well as pharmaceutically acceptable salts and derivatives (including prodrug forms) thereof where applicable, in context. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder. When the bond is shown, both a double bond and single bond are represented within the context of the compound shown.

The term “derivatives” can mean, but is in no way limited to, chemical compositions, for example, nucleic acids, nucleotides, polypeptides or amino acids, formed from the native compounds either directly, by modification, or by partial substitution. The term “analogs” can mean, but is in no way limited to, chemical compositions, for example, nucleic acids, nucleotides, polypeptides or amino acids that have a structure similar to, but not identical to, the native compound.

The term “effective amount/dose,” “pharmaceutically effective amount/dose,” “pharmaceutically effective amount/dose” or “therapeutically effective amount/dose” can mean, but is in no way limited to, that amount/dose of the active pharmaceutical ingredient sufficient to prevent, inhibit the occurrence, ameliorate, delay or treat (alleviate a symptom to some extent, preferably all) the symptoms of a condition, disorder or disease state. The effective amount depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 1000 mg/kg body weight/day of active ingredients is administered dependent upon potency of the agent. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present disclosure, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The term “pharmacological composition,” “therapeutic composition,” “therapeutic formulation” or “pharmaceutically acceptable formulation” can mean, but is in no way limited to, a composition or formulation that allows for the effective distribution of an agent provided by the present disclosure, which is in a form suitable for administration to the physical location most suitable for their desired activity, e.g., systemic administration.

The term “pharmaceutically acceptable” or “pharmacologically acceptable” can mean, but is in no way limited to, entities and compositions that do not produce an adverse, allergic or other untoward reaction when administered to an animal, or a human, as appropriate.

The term “pharmaceutically acceptable carrier” or “pharmacologically acceptable carrier” can mean, but is in no way limited to, any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The term “systemic administration” refers to a route of administration that is, e.g., enteral or parenteral, and results in the systemic distribution of an agent leading to systemic absorption or accumulation of drugs in the blood stream followed by distribution throughout the entire body. Suitable forms, in part, depend upon the use or the route of entry, for example oral, transdermal, or by injection. Such forms should not prevent the composition or formulation from reaching a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect. Administration routes which lead to systemic absorption include, without limitations: intravenous, subcutaneous, intraperitoneal, inhalation, oral, intrapulmonary and intramuscular. The rate of entry of a drug into the circulation has been shown to be a function of molecular weight or size. The use of a liposome or other drug carrier comprising the compounds of the present disclosure can potentially localize the drug, for example, in certain tissue types, such as the tissues of the reticular endothelial system (RES). A liposome formulation which can facilitate the association of drug with the surface of cells, such as, lymphocytes and macrophages is also useful.

The term “local administration” refers to a route of administration in which the agent is delivered to a site that is apposite or proximal, e.g., within about 10 cm, to the site of the lesion or disease.

The term “conservative mutations” refers to the substitution, deletion or addition of nucleic acids that alter, add or delete a single amino acid or a small number of amino acids in a coding sequence where the nucleic acid alterations result in the substitution of a chemically similar amino acid. Amino acids that may serve as conservative substitutions for each other include the following: Basic: Arginine (R), Lysine (K), Histidine (H); Acidic: Aspartic acid (D), Glutamic acid (E), Asparagine (N), Glutamine (Q); hydrophilic: Glycine (G), Alanine (A), Valine (V), Leucine (L), Isoleucine (I); Hydrophobic: Phenylalanine (F), Tyrosine (Y), Tryptophan (W); Sulfur-containing: Methionine (M), Cysteine (C). In addition, sequences that differ by conservative variations are generally homologous.

By “homology” is meant the nucleotide sequence of two or more nucleic acid molecules or two or more nucleic acid or amino acid sequences is partially or completely identical. In certain embodiments the homologous nucleic acid or amino acid sequence has 30%, 40%, 50%, 60%, 70%, 80%, 90%, or 95% sequence similarity or identity to an nucleic acid encoding the amino acid sequence of SEQ ID NO: 1, SEQ ID NO: 2, SEQ ID NO: 4, SEQ ID NO: 5, SEQ ID NO: 6, or SEQ ID NO: 7.

“Homologs” can be naturally occurring, or created by artificial synthesis of one or more nucleic acids having related sequences, or by modification of one or more nucleic acid to produce related nucleic acids. Nucleic acids are homologous when they are derived, naturally or artificially, from a common ancestor sequence (e.g., orthologs or paralogs). If the homology between two nucleic acids is not expressly described, homology can be inferred by a nucleic acid comparison between two or more sequences. If the sequences demonstrate some degree of sequence similarity, for example, greater than about 30% at the primary amino acid structure level, it is concluded that they share a common ancestor. For purposes of the present disclosure, genes are homologous if the nucleic acid sequences are sufficiently similar to allow recombination and/or hybridization under low stringency conditions. In addition, polypeptides are regarded as homologous if their nucleic acid sequences are sufficiently similar to allow recombination or hybridization under low stringency conditions, and optionally they demonstrate membrane repair activity, and optionally they can be recognized by (i.e., cross-react with) an antibody specific for an epitope contained within the amino acid sequence of at least one of SEQ ID NOs: 1-8.

The term “cell” can mean, but is in no way limited to, its usual biological sense, and does not refer to an entire multicellular organism. The cell can, for example, be in vivo, in vitro or ex vivo, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

The term “host cell” can mean, but is in no way limited to, a cell that might be used to carry a heterologous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce one or more of the polypeptide components of the present disclosure.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

As used herein, “P140 peptides” can mean but is not limited to phosphorylated peptides derived from the spliceosome U1-70K protein, including those exemplified in SEQ ID NOs.: 1, 2, 4, and 5. In certain instances, P140 is used to specifically refer to a peptide consisting of the amino acid sequence SEQ ID NO: 1, in which serine at position 10 is phosphorylated (e.g., SEQ ID NO: 4).

The term “therapeutically effective amount or dose” includes a dose of a drug that is capable of achieving a therapeutic effect in a subject in need thereof. For example, a therapeutically effective amount of a drug can be the amount that is capable of preventing or relieving one or more symptoms associated with a disease or disorder, e.g., tissue injury or muscle-related disease or disorder. The exact amount can be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

A kit is any manufacture (e.g. a package or container) comprising at least one reagent, e.g. a probe, for specifically detecting a marker of the present disclosure. The manufacture may be promoted, distributed, or sold as a unit for performing the methods of the present disclosure. The reagents included in such a kit comprise probes/primers and/or antibodies for use in detecting sensitivity and resistance gene expression. In addition, the kits of the present disclosure may preferably contain instructions which describe a suitable detection assay. Such kits can be conveniently used, e.g., in clinical settings, to diagnose patients exhibiting symptoms of cancer, in particular patients exhibiting the possible presence of a tumor.

All publications, patent applications, patents, and other references mentioned herein are incorporated by reference in their entirety. In the case of conflict, the present specification, including definitions, will control. In addition, the materials, methods, and examples are illustrative only and not intended to be limiting.

The following references, the entire disclosures of which are incorporated herein by reference, provide one of skill with a general definition of many of the terms used in the present disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, the Harper Collins Dictionary of Biology (1991).

Peptides, Compositions, and Formulations

The present description provides peptides and pharmaceutical compositions having the same for use in methods to treat, prevent, and/or ameliorate at least one symptom of virus-induced immunopathology, such as that observed in virus-induced pneumopathy or viral pneumonia. The present description also provides method that use the peptides and pharmaceutical compositions described herein to modulate the immune response (such as the immune response in the lung(s)) in a subject having a viral infection (such as viral pneumonia).

Thus, in one aspect the present description provides chemically modified peptides of SEQ ID NOs: 1, 2, 4, 5, 6, and 7, including derivatives, analogs and salt forms thereof.

In any aspect or embodiment described herein, the description provides an isolated peptide comprising or consisting of the amino acid sequence of SEQ ID NO: 1:

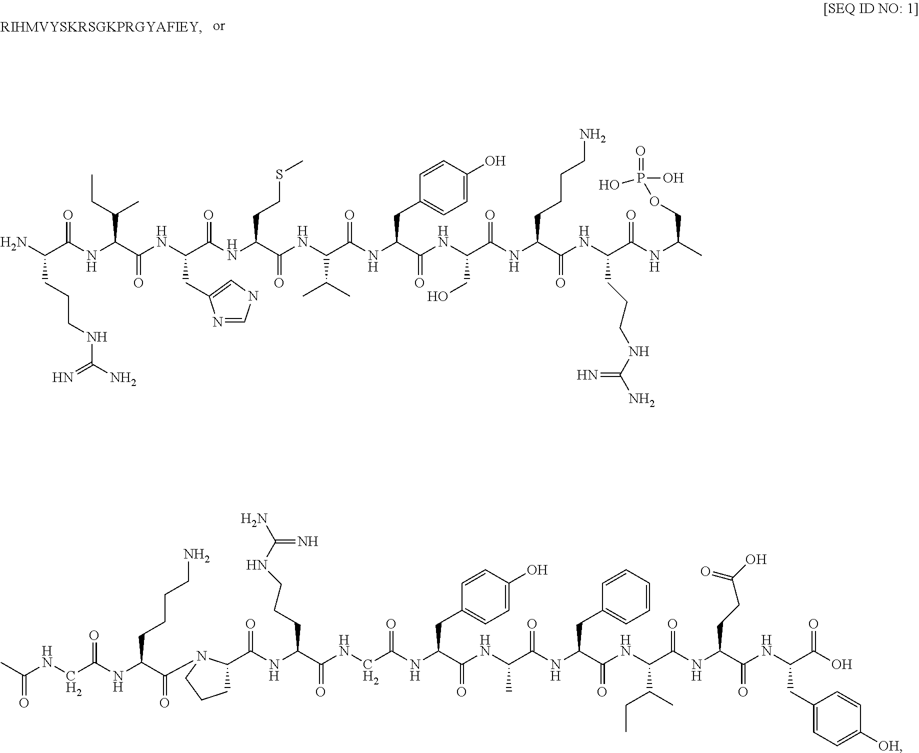

or salt thereof, having at least one post-translational modification selected from the group consisting of phosphorylation of a serine residue, oxidation of a methionine residue, and acetylation of a lysine residue, and combinations thereof. In any aspect or embodiment described herein, the description provides a composition or formulation comprising an isolated and/or chemically modified peptide (recombinant or synthesized) having or consisting of the amino acid sequence as set forth in SEQ ID NO: 1, or salt thereof, wherein the peptide comprises a phosphoserine at position 10 [SEQ ID NO: 4]. In any aspect or embodiment described herein, the description provides an isolated and/or chemically modified peptide (recombinant or synthesized) having or consisting of the amino acid sequence as set forth in SEQ ID NO: 1, or salt thereof, wherein the peptide comprises a phosphoserine at position 10, and an oxidized Methionine residue at position 4 [SEQ ID NO: 6]. In any aspect or embodiment described herein, the description provides a composition or formulation comprising an isolated and/or chemically modified peptide (recombinant or synthesized) having or consisting of the amino acid sequence as set forth in SEQ ID NO: 1, or salt thereof, wherein the peptide comprises a phosphoserine at position 10, and an oxidized Methionine residue at position 4 [SEQ ID NO: 6].

In any aspect or embodiment described herein, the peptide as set forth in SEQ ID NO: 1, 4, or 6 further comprises an acetylated lysine residue. In particular, said peptide as set forth in SEQ ID NO: 1 comprises a phosphoserine at position 10, and an oxidized Methionine residue at position 4, and an acetylation of one or both of the lysine at position 8 and 12, and more particularly further comprises a phosphoserine at position 7.

In any aspect or embodiment described herein, the description provides an isolated and/or chemically modified peptide (recombinant or synthesized), or a salt thereof, comprising or consisting of the amino acid sequence: IHMVYSKRSGKPRGYAFIEY [SEQ ID NO: 2], in which the Serine (S) at position 9 is phosphorylated [SEQ ID NO: 5]. In any aspect or embodiment described herein, description provides an isolated and/or chemically modified peptide (recombinant or synthesized), or a salt thereof, comprising or consisting of the amino acid sequence:

[SEQ ID NO: 2]
IHMVYSKRSGKPRGYAFIEY,

in which the Serine (S) at position 9 is phosphorylated and the Methionine (M) at position 3 is oxidized [SEQ ID NO: 7].

In any aspect or embodiment described herein, the description provides a peptide of Compound I having the following formula:

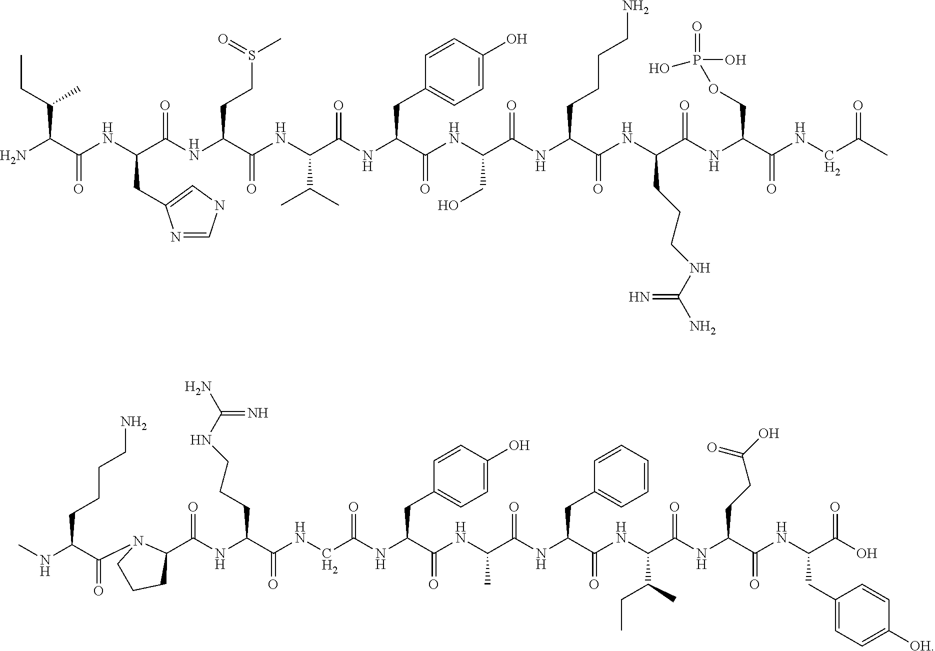

Compound I can also be represented by:

[SEQ ID NO: 5]
IHM(O)VYSKRS(PO₃H₂)GKPRGYAFIEY,

in which “M(O)” represents oxidized methionine, and “S(PO₃H₂)” represents phosphoserine.

Peptides of the present disclosure are derived from the human U1 snRNP 70 kDa protein (SEQ ID NO: 3), and correspond to the region delimited by the amino acid segment extending from the residue 132 to the residue 151 of SEQ ID NO: 3. Formally, the residue which is phosphorylated corresponds to the amino acid at the position 140 from the first methionine of SEQ ID NO: 3, and the residue which is optionally oxidized corresponds to the amino acid at the position 134 from the first methionine of SEQ ID NO: 3.

In any aspect or embodiment described herein, the description provides an isolated and/or chemically modified peptide (recombinant or synthesized) comprising or consisting of the amino acid sequence as set forth in SEQ ID NO: 1, or salt thereof, having at least one post-translational modification selected from the group consisting of phosphorylation of a serine residue, oxidation of a methionine residue, and acetylation of a lysine residue, and combinations thereof. In any aspect or embodiment described herein, the description provides a composition or formulation comprising an isolated peptide having or consisting of the amino acid sequence as set forth in SEQ ID NO: 1, or salt thereof, wherein the peptide comprises a phosphoserine at position 10 [SEQ ID NO: 4]. In any aspect or embodiment described herein, SEQ ID NO: 1 also comprises an oxidized Methionine residue at position 4 [SEQ ID NO: 6]. In any aspect or embodiment described herein, the peptide comprising or consisting of the amino acid sequence as set forth in SEQ ID NO: 4 or SEQ ID NO: 6 also comprises an acetylated lysine residue.

In any aspect or embodiment described herein, the description provides an isolated and/or chemically modified peptide (recombinant or synthesized) comprising or consisting of the amino acid sequence as set forth in SEQ ID NO: 2, or salt thereof, having at least one post-translational modification selected from the group consisting of phosphorylation of a serine residue, oxidation of a methionine residue, and acetylation of a lysine residue, and combinations thereof. In any aspect or embodiment described herein, the description provides a composition or formulation comprising an isolated and/or chemically modified peptide (recombinant or synthesized) having or consisting of the amino acid sequence of SEQ ID NO: 2, or salt thereof, wherein the peptide comprises a phosphoserine at position 9 [SEQ ID NO: 5]. In any aspect or embodiment described herein, the description provides a composition or formulation comprising an isolated and/or chemically modified peptide (recombinant or synthesized) having or consisting of the amino acid sequence of SEQ ID NO: 2, or salt thereof, wherein the peptide comprises a phosphoserine at position 9 and an oxidized Methionine residue at position 3 [SEQ ID NO: 7]. In any aspect or embodiment described herein, the peptide comprising or consisting of the amino acid sequence as set forth in SEQ ID NO: 5 or SEQ ID NO: 7 also comprises an acetylated lysine residue.

In any aspect or embodiment described herein, the description provides a peptide of Compound II having the following formula:

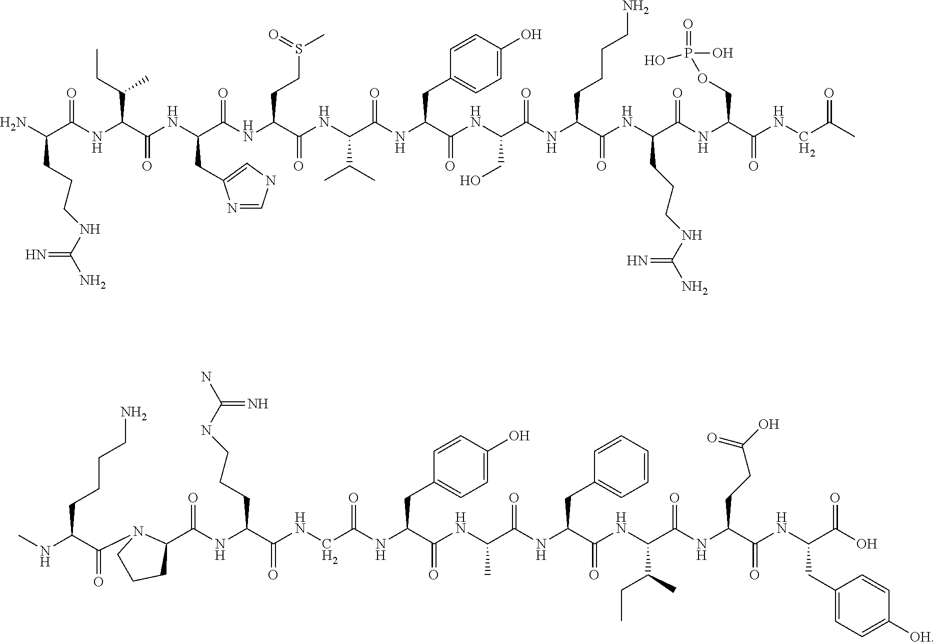

Compound II can also be represented by:

[SEQ ID NO: 6]
RIHM(O)VYSKRS(PO₃H₂)GKPRGYAFIEY,

in which M(O) represents oxidation of methionine, and S(PO₃H₂) represents the phosphorylation of serine.

Thus, the description provides peptides, or a salt thereof, comprising or consisting of the amino acid sequence chosen among the group consisting of SEQ ID NO: 6 and SEQ ID NO: 7. The present description also provides compositions and formulations comprising peptides, or a salt thereof, comprising or consisting of the amino acid sequence chosen among the group consisting of SEQ ID NO: 6 and SEQ ID NO: 7.

In any aspect or embodiment described herein, the description provides a composition or formulation comprising an effective amount of at least one peptide, or salt thereof, selected from the group consisting of the amino acid sequence SEQ ID NO: 2, or salt thereof, comprising a phosphoserine at position 9 and oxidized Methionine at position 3 [SEQ ID NO: 7]; amino acid sequence of SEQ ID NO: 1, or salt thereof, comprising a phosphoserine at position 10 [SEQ ID NO: 4]; the amino acid sequence SEQ ID NO: 1, or salt thereof, comprising a phosphoserine at position 10 and an oxidized Methionine at position 4 [SEQ ID NO: 6]; and a combination thereof.

The description provides peptides, and/or salts thereof, comprising or consisting of the amino acid sequence chosen among the group consisting of SEQ ID NO: 1, 2, 4, 5, 6, 7, and combinations thereof, as well as compositions and formulations comprising the same.

In any aspect or embodiment described herein, the description provides an isolated and/or chemically modified peptide (recombinant or synthesized) having the amino acid sequence of SEQ ID NO: 1, comprising a phosphoserine at position 10 [SEQ ID NO: 4]. In any aspect or embodiment described herein, the P140 peptides also comprises an oxidized methionine at position 4 (e.g., SEQ ID NO: 6) (herein, also referred to as Compound II or P140(MO)). In any aspect or embodiment described herein, the description provides the peptide having the amino acid sequence as set forth in SEQ ID NO: 1, comprising a phosphoserine at position 10 and an oxidized methionine at position 4 [SEQ ID NO: 6], or salt thereof, and an effective amount of a carrier, e.g., a pharmaceutically acceptable carrier. In certain additional embodiments, the description provides a composition, e.g., a therapeutic composition, comprising an effective amount of a peptide having the amino acid sequence as set forth in SEQ ID NO: 1, comprising a phosphoserine at position 10 and an oxidized methionine at position 4 [SEQ ID NO: 6], or salt thereof, and an effective amount of a carrier, e.g., a pharmaceutically acceptable carrier.

According to the present description, the isolated and/or chemically modified peptide (recombinant or synthesized) having the amino acid sequence of SEQ ID NO: 1 or 2 is modified by at least one post-translational modification (modifications that occur after the synthesis of the peptides) (e.g., a peptide having an amino acid sequence as set forth in SEQ ID NO: 4, 5, 6, or 7). In any aspect or embodiment described herein, the post-translational modification is selected from the group consisting of phosphorylation (addition of a phosphate PO₃H₂), e.g., phosphorylation of a serine residue; oxidation, e.g., oxidation of a methionine residue; acetylation, e.g., acetylation of a lysine residue; and combinations thereof. In certain embodiments, the isolated and/or chemically modified peptide (recombinant or synthesized) having the amino acid sequence of SEQ ID NO: 1 or 2 is modified by at least two post-translational modifications (e.g., a peptide having an amino acid sequence as set forth in SEQ ID NO: 4, 5, 6, or 7).

In any aspect or embodiment described herein, the description provides an isolated and/or chemically modified peptide (recombinant or synthesized) having the amino acid sequence as set forth in SEQ ID NO: 1 comprising a phosphoserine at position 10 [e.g., SEQ ID NO: 4], or salt thereof. In any aspect or embodiment described herein, the description provides compositions and formulations comprising a peptide having the amino acid sequence as set forth in SEQ ID NO: 1, comprising a phosphoserine at position 10 [e.g., SEQ ID NO: 4], or salt thereof. In any aspect or embodiment described herein, the composition or formulation of the present disclosure further comprises at least one of: an effective amount of a carrier (e.g., a pharmaceutically acceptable carrier), an effective amount of an excipient (e.g., a pharmaceutically acceptable excipient), or combinations thereof.

In any aspect or embodiment described herein, the description provides an isolated and/or chemically modified peptide (recombinant or synthesized) having the amino acid sequence as set forth in SEQ ID NO: 2 comprising a phosphoserine at position 9 [e.g., SEQ ID NO: 5], or salt thereof. In any aspect or embodiment described herein, the description provides compositions and formulations comprising a peptide having the amino acid sequence as set forth in SEQ ID NO: 2, comprising a phosphoserine at position 9 [e.g., SEQ ID NO: 5], or salt thereof. In any aspect or embodiment described herein, any of compositions or formulations of the present disclosure further comprises at least one of: an effective amount of a carrier (e.g., a pharmaceutically acceptable carrier), an effective amount of an excipient (e.g., a pharmaceutically acceptable excipient), or combinations thereof.

In any aspect or embodiment described herein, the description provides an isolated and/or chemically modified peptide (recombinant or synthesized) having the amino acid sequence as set forth in SEQ ID NO: 1 comprising a phosphoserine at position 10 and an oxidized methionine at position 4 [e.g., SEQ ID NO: 6], or salt thereof. In any aspect or embodiment described herein, the description provides compositions or formulations comprising an effective amount of a peptide having the amino acid sequence as set forth in SEQ ID NO: 1, comprising a phosphoserine at position 10 and an oxidized methionine at position 4 [e.g., SEQ ID NO: 6], or salt thereof.

In any aspect or embodiment described herein, the description provides an isolated and/or chemically modified peptide (recombinant or synthesized) having the amino acid sequence as set forth in SEQ ID NO: 2 comprising a phosphoserine at position 9 and an oxidized methionine at position 3 [e.g., SEQ ID NO: 7], or salt thereof. In any aspect or embodiment described herein, the description provides compositions and formulations comprising a peptide having the amino acid sequence as set forth in SEQ ID NO: 2, comprising a phosphoserine at position 9 and an oxidized methionine at position 3, or salt thereof [e.g., SEQ ID NO: 7]. In any aspect or embodiment described herein, the composition or formulation of the present disclosure further comprises at least one of: an effective amount of a carrier (e.g., a pharmaceutically acceptable carrier), an effective amount of an excipient (e.g., a pharmaceutically acceptable excipient), or combinations thereof.

In any aspect or embodiment described herein, the description provides a pharmaceutical composition comprising one or more peptides of the present disclosure. For example, in any aspect or embodiment described herein the pharmaceutical composition or formulation comprises: one or more peptides of the present disclosure, and one or more of: an effective amount of a carrier (e.g., a pharmaceutically acceptable carrier), an effective amount of an excipient (e.g., a pharmaceutically acceptable excipient), or combinations thereof. In any aspect or embodiment described herein, the composition comprises an effective amount of a peptide having the amino acid sequence as set forth in SEQ ID NO: 4, SEQ ID NO: 5, SEQ ID NO: 6, SEQ ID NO: 7, salt form thereof, or a combination thereof. In any aspect or embodiment described herein, the composition consists essentially of an effective amount of a peptide having the amino acid sequence as set forth in SEQ ID NO: 4, SEQ ID NO: 5, SEQ ID NO: 6, SEQ ID NO: 7, salt form thereof, or a combination thereof. In any aspect or embodiment described herein, the composition consists of an effective amount of a peptide having the amino acid sequence as set forth in SEQ ID NO: 4, SEQ ID NO: 5, SEQ ID NO: 6, SEQ ID NO: 7, salt form thereof, or a combination thereof.

Surprisingly and unexpectedly, it was discovered that the peptides as described herein are more stable in vitro compared to the non-oxidized counterpart. The stability is measured as disclosed in the example section. The phosphorylated-oxidized peptide is less spontaneously degraded in solution compared to the non-oxidized counterpart, said stability enhancing its biological properties. In addition, the inventors have surprisingly identified that the methionine oxidation enhances the peptide stability, without affecting the biological effect of such peptide, contrary to the teaching of the prior art. Indeed, it is largely reported in the art that proteins or peptides containing oxidized methionine have disruptions in their three-dimensional structure and/or bioactivity. The modified peptides as described herein have an affinity for HSC70 protein essentially identical to the non-oxidized counterpart as disclosed in the example section.

In certain embodiments, the oxidation occurs in the Methionine (M) at position 9 of SEQ ID NO: 2, or at position 10 of SEQ ID NO: 1, which are the equivalent to position 134 of SEQ ID NO: 3. The sulfur atom is oxidized as illustrated below:

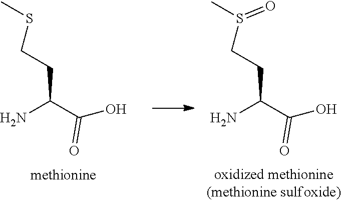

The above peptides (SEQ ID NO: 1, 2, 4, 5, 6, and 7) can be synthesized by techniques commonly used in the art, such as biological synthesis or chemical synthesis. Biological synthesis refers to the production, in vivo, in vitro or ex vivo, of the peptide of interest, by the transcription and translation of a nucleic acid molecule coding for said peptides.

For instance the nucleic acid sequence:

[SEQ ID NO: 8]
MGNATHCAYATGGTNTAYWSNAARMGNWSNGGNAARCCNMGNGGNTAY
GCNTTYATHGARTAYTRR

is transcribed and translated either in an in vitro system, or in a host organism, in order to produce the peptide SEQ ID NO: 1. The produced peptide is thus purified according to well-known techniques.

Chemical synthesis consists to polymerize the desired peptide by adding the required amino acids. A method is disclosed in the example section.

It is possible to chemically synthesize the peptides SEQ ID NO: 1 and 2 by classical Fmoc (N-[9-fluorenyl] methoxycarbonyl) solid-phase chemistry and purified by reversed-phase high-performance liquid chromatography (HPLC; Neimark and Briand, 1993; Monneaux et al., 2003, Eur. J. Immunol. 33, 287-296; Page et al., 2009, PloS ONE 4, e5273).

It is also possible to directly synthesize the peptides SEQ ID NO: 1 and 2, in which respective residues at position 10 and 9 are phosphorylated. For this purpose, during the peptide synthesis a Fmoc-Ser(PO(Obz)OH)-OH-type serine derivative was used, at the desired position.

Phosphate group (—PO₃H₂) can also be added after the synthesis of the peptide, according to protocols well known in the art.

Serine can be phosphorylated by incubating the peptides SEQ ID NO: 1 or 2 with specific serine kinase chosen among Protein Kinase A or C (PKA or PKC) or casein kinase II, in presence of adenosine triphosphate (ATP). The peptides are thus phosphorylated in one serine (at position 6 or 9 of SEQ ID NO: 2, or at position 7 or 10 of SEQ ID NO: 1), or both serine. The desired phosphorylated peptide is separated from the others for instance by chromatography.

A chemical addition of —PO₃H₂ can also be added at the specific position (at position 9 of SEQ ID NO: 2, or at position 10 of SEQ ID NO: 1), by using specific protective group, that the skilled person can easily choose according to his common knowledge. Any other techniques known in the art, allowing the specific phosphorylation of serine, can be used.

In certain embodiments, the oxidation of Methionine is performed according to the following process: treating with either with H₂O₂, 20 mM, at 37° C. for 4 hours, or in a solution of dimethylsulfoxyde (DMSO; Me₂SO), 0.1M plus HCl 0.5 M, at 22° C. for 30 to 180 minutes. Any other techniques known in the art, allowing the specific oxidation of methionine, can be used.

In any of the aspects or embodiments described herein, the peptide(s) provided by the description can be present in a form of a salt known to a person skilled in the art, such as, e.g., sodium salts, ammonium salts, calcium salts, magnesium salts, potassium salts, acetate salts, carbonate salts, citrate salts, chloride salts, sulphate salts, amino chlorhydate salts, borhydrate salts, benzensulphonate salts, phosphate salts, dihydrogenophosphate salts, succinate salts, citrate salts, tartrate salts, lactate salts, mandelate salts, methane sulfonate salts (mesylate) or p-toluene sulfonate salts (tosylate). This list is provided by way of example and is not meant to be limiting on the present disclosure. For example, the skilled person can easily determine, according to his knowledge, the appropriate salt.

In any aspect or embodiment described herein, the description provides a peptide comprising or consisting of the amino acid sequence:

[SEQ ID NO: 1]
RIHMVYSKRSGKPRGYAFIEY,

comprising a phosphoserine at position 10 [e.g., SEQ ID NO: 4]. In certain embodiments, the phosphorylated peptide further comprises an oxidized Methionine at position 4, or salt thereof [e.g., SEQ ID NO: 6]. In one advantageous embodiment, the peptide, as defined above, consist of the amino acid sequence SEQ ID NO: 4, or salt thereof. In one advantageous embodiment, the peptide, as defined above, consists of the amino acid sequence SEQ ID NO: 6, or salt thereof.

In any aspect or embodiments described herein, the pharmaceutical compositions or formulations described herein further comprises an effective amount of an excipient or carrier (e.g., an effective amount of a pharmaceutically acceptable carrier). As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The description provides methods for preparing pharmaceutical compositions or formulations. Such methods comprise formulating an effective amount of a pharmaceutically acceptable carrier with one or more peptides as described herein. Such compositions or formulations can further include additional active agents as described above. Thus, the present disclosure further describes methods for preparing a pharmaceutical composition or formulation.

A pharmaceutical composition or formulation of the present disclosure is formulated to be compatible with its intended route of administration. Examples of routes of administration include parenteral, e.g., intravenous, intradermal, subcutaneous, oral, nasal (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol; antioxidants such as ascorbic acid or sodium bisulfate; chelating agents such as ethylenediamine-tetraacetic acid; buffers such as acetates, citrates or phosphates and agents for the adjustment of tonicity such as sodium chloride or dextrubinrubi. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions or formulations suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition or formulation must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, chlorobutanol, phenol, ascorbic acid, and the like. In many cases, it will be preferable to include isotonic agents, for example, sugars, polyalcohols such as mannitol, sorbitol, or sodium chloride in the composition or formulation. Prolonged absorption of the injectable compositions or formulations can be brought about by including in the composition or formulation an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions or formulations generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions or formulations can also be prepared using a fluid carrier for use as a mouthwash, wherein the compound in the fluid carrier is applied orally and swished and expectorated or swallowed

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In any aspect or embodiment described herein, the active compounds are prepared with carriers in an amount that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions or formulations in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the present disclosure are dictated by and directly dependent on the unique characteristics of the active compound and the particular therapeutic effect to be achieved, and the limitations inherent in the art of compounding such an active compound for the treatment of individuals.

In any aspect or embodiment of the methods provided herein, the method can further include the step of administering a dosage from about 100 ng to about 5 mg of a therapeutic or pharmaceutical composition or formulation as described herein. In any aspect or embodiment described herein, e.g., in human, the pharmaceutical composition or formulation as described herein may contain mannitol as carrier, and the composition or formulation is administered from 10 μg to 500 μg, preferably 200 μg, in a single administration.

In certain additional aspects, the dosage regimen can be reproduced from 1 to 3 times/week, every week to every four week for as long as needed with therapeutic windows and thus for several years. In a preferred embodiment, the dosage regimen is once every 4 weeks of treatment but can be repeated twice a year for several years. An example of administration is: one injection of 200 μg of peptide, every 4 weeks, for 12 weeks (i.e. 3 injections separated from each other by 4 weeks). The treatment can be prolonged by administration every 6 months.

Preferred pharmaceutically acceptable carriers can comprise, for example, xanthan gum, locust bean gum, galactose, other saccharides, oligosaccharides and/or polysaccharides, starch, starch fragments, dextrins, British gum and mixtures thereof. Advantageously, the pharmaceutically acceptable carrier is of natural origin. The pharmaceutically acceptable carrier can be, or can further comprise, an inert saccharide diluent selected from a monosaccharide or disaccharide. Advantageous saccharide is mannitol.

Advantageously, the present disclosure relates to a pharmaceutical composition or formulation as defined above, which is in the form of a liposome, or nano particles, or in the form of a solution. An advantageous solution is a solution comprising from 1 to 15%, in particular about 10% of mannitol. The solution should be iso-osmolar.

The present disclosure also relates to a drug comprising a combination product as defined above, for a simultaneous, separate or sequential use.

Therapeutic Methods

A further aspect of the present disclosure provides a method of treating, preventing or ameliorating at least one symptom of virus-induced immunopathology (e.g., virus-induced immunopathology observed in virus-pneumopathy or viral pneumonia) in a subject in need thereof. The method comprises: providing a subject in need thereof; and administering an effective amount of the pharmaceutical composition or formulation described herein, wherein the peptide effectuates the prevention, treatment, or amelioration of at least one symptom of the virus-induced immunopathology (e.g., at least one symptom of virus-pneumopathy or viral pneumonia).

In an additional aspect, the present description provides methods for modulating the immune response (such as the immune response in the lung(s)) in a subject having viral infection (e.g., a subject having viral pneumonia), the method comprising: providing a subject in need thereof; and administering an effective amount of one or more peptides of the present disclosure or a pharmaceutical composition of the present disclosure, wherein modulating the immune response is effective to treat, prevent, or ameliorate at least one symptom of virus-induced immunopathology (e.g., at least one symptom of virus-induced pneumopathy or viral pneumonia).

In any aspect or embodiment described herein, the viral infection is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, LCMV (lymphocytic choriomeningitis virus), hepatitis B virus, Coxsackie B virus (CBV), Human Immunodeficiency Virus (HIV), Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof.

In any aspect or embodiment described herein, the virus-induced immunopathology, the virus-induced pneumopathy, or viral pneumonia is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, LCMV (lymphocytic choriomeningitis virus), hepatitis B virus, Coxsackie B virus (CBV), Human Immunodeficiency Virus (HIV), Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof. For example, in any aspect or embodiment described herein, the virus-induced immunopathology, the viral pneumopathy, or viral pneumonia is related to at least one disease selected from Middle East Respiratory Syndrome (MERS), Severe Acute Respiratory Syndrome (SARS), Coronavirus Disease 2019 (COVID-19), or a combination thereof.

In any aspect or embodiment described herein, the viral infection is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof.

In any aspect or embodiment described herein, the virus-induced immunopathology, the virus-induced pneumopathy, or viral pneumonia is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof. For example, in any aspect or embodiment described herein, the virus-induced immunopathology, the viral pneumopathy, or viral pneumonia is related to at least one disease selected from Middle East Respiratory Syndrome (MERS), Severe Acute Respiratory Syndrome (SARS), Coronavirus Disease 2019 (COVID-19), or a combination thereof.

In any aspect or embodiment described herein, the method treats, prevents, or ameliorates at least one symptom of COVID-19.

In any aspect or embodiment described herein, the method treats, prevents, or ameliorates COVID-19 pathology.

### EXAMPLES

### Example 1: Chemical Synthesis of the Peptides

P140 peptide and P140(MO) were synthesized using classical Fmoc (N-[9-fluorenyl] methoxycarbonyl) solid-phase chemistry and purified by reversed-phase high-performance liquid chromatography (HPLC; Neimark and Briand, 1993; Monneaux et al., 2003, Eur. J. Immunol. 33, 287-296; Page et al., 2009, PloS ONE 4, e5273). Their homogeneity was checked by analytical HPLC, and their identity was assessed by LC/MS on a Finnigan LCQ Advantage Max system (Thermo Fischer Scientific). After completion of the reaction, the peptides were purified by HPLC.

In order to introduce the phosphorylation at the serine residue equivalent to the residue 140 of SEQ ID NO: 3, an Fmoc-Ser(PO(Obz)OH)—OH-type serine derivative was used. The coupling time is increased to 30 minutes and a second coupling is carried out systematically. After cleavage in acid medium, each peptide is precipitated by cold ether, solubilized in a solution of water and acetonitrile and finally lyophilized. The peptides are then purified by RP-HPLC, their integrity and their purity has been analyzed by analytic HPLC and by mass spectrometry (Maldi-TOF). Oxidation is introduced as mentioned above.

### Example 2: Stability of the Peptides

The stability of the peptide SEQ ID NO: 1 in which the serine at position 10 is phosphorylated and the methionine at position 4 is oxydized (P140(MO)), and the peptide SEQ ID NO: 1 in which the serine at position 10 is phosphorylated (P140) was measured at 37° C., in a solution of 10% (v/v) mannitol. For each peptide, 3 concentrations have been tested: 200, 100 and 50 μg/mL.

At the indicated time, the integrity of P140 and P140(MO) peptides was measured in saline by high-performance liquid chromatography from the area of the peak corresponding to the intact peptide.

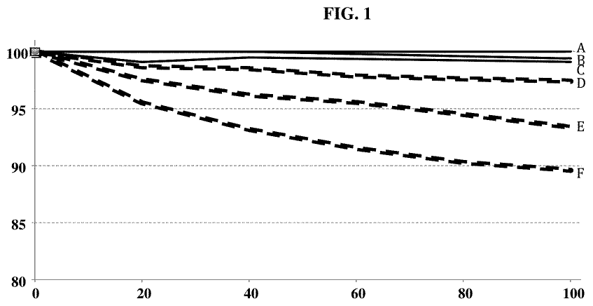

Results are shown in FIG. 1.

The following tables 1 and 2 summarize the results:

TABLE 1

| | | P140 (MO) | | | P140 | | |
|---|---|---|---|---|---|---|---|
| Stability | Days | 200 μg/mL | 100 μg/mL | 50 μg/mL | 200 μg/mL | 100 μg/mL | 50 μg/mL |
| (%) | 0 | 100 | 100 | 100 | 100 | 100 | 100 |
| | 20 | 100 | 99.1 | 100 | 98.7 | 97.5 | 95.5 |
| | 40 | 100 | 99.5 | 100 | 98.5 | 96.2 | 93.2 |
| | 60 | — | — | — | 97.9 | 95.5 | 91.5 |
| | 80 | — | — | — | 97.6 | 94.5 | 90.3 |
| | 100 | 100 | 99.1 | 99.4 | 97.4 | 93.4 | 89.6 |

TABLE 2

| | | P140 (MO) | | | P140 | | |
|---|---|---|---|---|---|---|---|
| Stability | Days | 200 μg/mL | 100 μg/mL | 50 μg/mL | 200 μg/mL | 100 μg/mL | 50 μg/mL |
| (%) | Linear | y = 100 | y = | y = | y = | y = | y = |
| | equation | | −0.0064x + | −0.0064x + | −0.0238x + | −0.0612x + | −0.099x + |
| | | | 100.11 | 99.677 | 99.535 | 99.25 | 98.299 |
| | Correlation | N/A | R² = | R² = | R² = | R² = | R² = |
| | coefficient | | 0.8571 | 0.4157 | 0.8854 | 0.9538 | 0.9065 |
| | 95% of | ∞ | 2 years + | 2 years | 6 months | 2 months | 1 months |
| | stability | | 2 months | | | | |
| | (predicted) | | | | | | |

Stability is measured by using the HPLC peak surface.

P140 M(O) stability remains unchanged (100%, 99.1% and 99.4%)over 100 days at 37° C., for each of the tested concentrations (50 to 200 μg/ml).

P140 stability decreases over the time and is reduced after 100 days at 37° C. (97.4%, 93.4% et 89.6%) for each of the tested concentrations (50 to 200 μg/ml).

These data demonstrate that the oxidation of the methionine in the peptide P140 enhance the stability of the peptide. P140(MO) is stable at all the tested concentration over 100 days.

### Example 3: Therapeutic Effect of the Peptides in MRL/lpr Mice

MRL/lpr mouse strain is a mouse substrain that is genetically predisposed to the development of systemic lupus erythematosus-like syndrome, which has been found to be clinically similar to the human disease. It has been determined that this mouse strain carries a mutation in the fas gene. Also, the MRL/lpr is a useful model to study behavioural and cognitive deficits found in autoimmune diseases and the efficacy of immunosuppressive agents [Monneaux et al., 2003, Eur. J. Immunol. 33, 287-296].

2.1—Survival Analysis

Five-week-old female MRL/lpr mice received P140 or peptide P140(MO) intravenously as described (Monneaux et al., 2003, Eur. J. Immunol. 33, 287-296). All experimental protocols were carried out with the approval of the local Institutional Animal Care and Use Committee (CREMEAS). As control, mice were injected with NaCl.

Twenty mice were used for each peptide or NaCl.

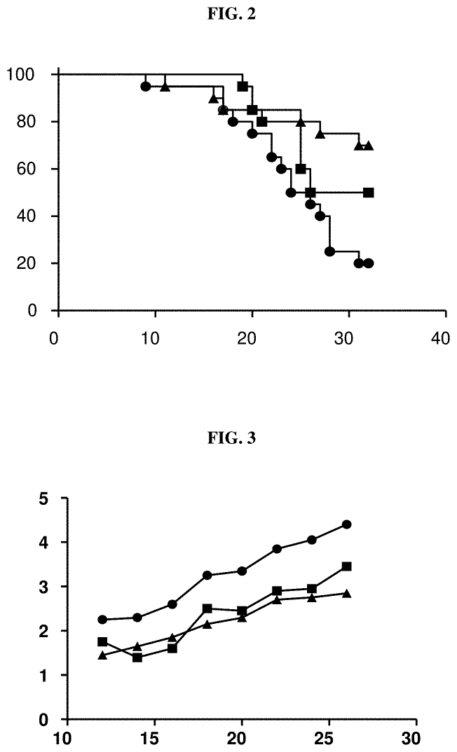

The results are shown in FIG. 2.

A Log-rank (Mantel-Cox) Test has been applied and the results are the following: NaCl vs P140 p=0.0686, NaCl vs P140(MO) p=0.0026, P140 vs P140 M(O) p=0.2366.

The Median survival of mice is: NaCl=25 weeks, P140=29 weeks and P140 (MO)>40 weeks. These results demonstrate the efficacy of the P140(MO) peptide in vivo in the treatment of lupus, in mice.

2.2—Proteinuria Analysis

Proteinuria of the above mice was measured in fresh urine using Albustix (Bayer Diagnostics) and was semi-quantitatively estimated according to a 0-4 scale recommended by the manufacturer (no proteinuria=0; traces=1; 1+=2; 2+=3; 3+=4; 4+=5).

The results are shown in FIG. 3.

In this figure, it is observed that the proteinuria is less important and appears lately in P140 M(O)-treated mice compared to the untreated mice.

2.3—Cellularity Analysis

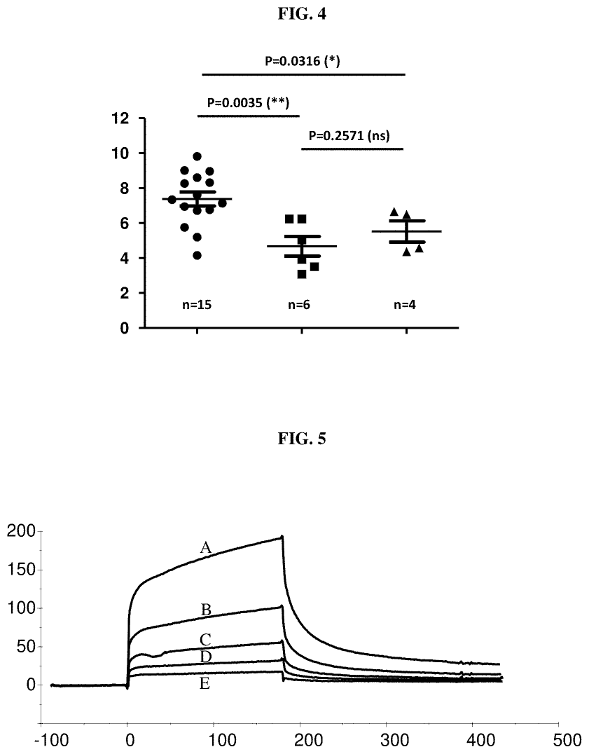

MRL/lpr mice were injected with 100 μg/100 μL of P140 or P140(MO) and cellularity (preipheral blood) was studied 5 days after this unique injection. The count includes all the leucocytes. In view of the low number of tested mice, a non parametric statistical test has been realised Mann-Whitney). The results are shown in FIG. 4.

Thus, in an acute murine model of lupus, peptide of SEQ ID NO: 6 was able to decrease peripheral hypercellularity and delays biological and clinical signs of the disease with an efficacy at least similar to that of P140, or better.

Statistics

Statistical tests were performed using GraphPad Prism version 5.0. The two-way ANOVA test was used to analyze statistical significance of proteinuria differences between control and peptide-treated groups of mice. Survival of control and P140 analogue-treated female MRL/lpr mice was analyzed by the Kaplan-Meier method, and the significance of differences was determined by the log-rank test. For the other variables, statistical significance was assessed using the Student's t-test. p values less than 0.05 were considered significant.

### Example 4: Affinity of the Peptides for HSC70 Protein

BIAcore 3000 system (Biacore AB) was used to evaluate the binding of P140 peptides to HSC70 protein (Page et al., 2009, and 2011). Sensor chip CM5, surfactant P20, amine coupling kit containing N-hydroxysuccinimide (NHS) and N-ethyl-N′-dimethylaminopropyl carbodiimide (EDC), 2-(2-pyridinyldithio)ethaneamine (PDEA) and ethanolamine were from Biacore AB. Biosensor assays were performed with HBS-EP buffer as running buffer (10 mM HEPES, 150 mM NaCl, 3 mM EDTA, 0.005% surfactant P20, pH 7.4). The compounds were diluted in the running buffer. The sensor chip surface was regenerated after each experiment by injecting 10 μL of 10 mM HCl. Recombinant bovine HSC70 (Stressgen) was immobilized on flow cells of a CM5 sensor chip through its thiol groups using 35 μL PDEA in 50 mM borate buffer, pH 8.3 on the NHS/EDC-activated matrix. Then, 35 μL of HSC70 (100 μg/mL in formate buffer, pH 4.3) were injected until a response of 13,000 response units (RU) corresponding to 13 ng/mm² of HSC70 was immobilized. Twenty μL of a 50 mM cysteine/1 M NaCl solution was used to saturate unoccupied sites on the chip. The direct binding measurement of P140 peptides to HSC70 was carried out at 25° C. with a constant flow rate of 20 μL/min. P140 peptide and analogues were injected in the flux at different concentrations for 3 min, followed by a dissociation phase of 3 min. The kinetic parameters were calculated using the BIAeval 3.1 software on a personal computer. Analysis was performed using the simple 1:1 Langmuir binding model. The specific binding profiles were obtained after subtracting the response signal from the control empty channel and from blank-buffer injection. The fitting to each model was judged by the χ² value and randomness of residue distribution compared to the theoretical model.

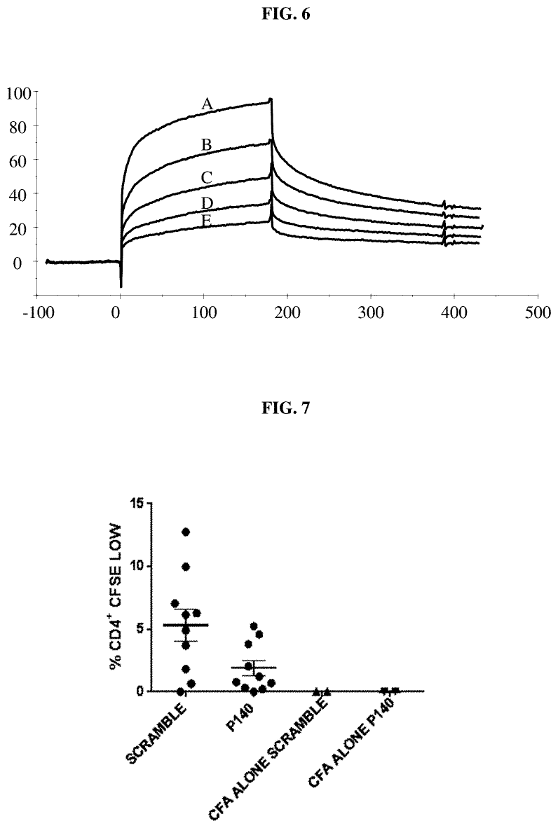

Results are shown in Tables 3 and 4, and in FIGS. 5 and 6.

These tables demonstrate that the affinity for HSC70 is not statistically different between P140 and P140 M(O) peptides.

Thus, these two peptides bind with the same efficiency HSC70.

### Example 5: Effect of P140 Peptide in RA

In this example, a P140 peptide (21-mer linear peptide) encompassing the sequence 131-151 of the spliceosomal U1-70K protein and containing a phosphoserine residue at position 140, was tested. After P140 treatment, an accumulation of autophagy markers SQSTM1 and MAP1LC3 was observed in MRL/lpr B cells, consistent with a down-regulation of autophagic flux (Page et al., 2011). Chaperone-mediated autophagy (CMA) was also found to be a target of P140 peptide and it was demonstrated that P140 peptide inhibitory effect on CMA is likely tied to its ability to interact with HSPA8 chaperone protein (Page et al., 2009) and to alter the composition of HSPA8 heterocomplexes (Macri et al., in press). Expression of both HSPA8 and the limiting CMA component LAMP-2A, which is increased in MRL/lpr B cells, is down-regulated after treating mice with P140 peptide (Page et al., 2011; Macri et al., in press). It was shown further that P140, but not the non-phosphorylated peptide that is not protective against disease development in mice (Monneaux et al., 2003), uses the clathrin-dependent endo-lysosomal pathway to enter into MRL/lpr B lymphocytes and accumulates in the lysosomal lumen where it may directly hamper lysosomal HSPA8 chaperoning functions, and also destabilize LAMP-2A in lysosomes as a result of its effect on HSP90 (Macri et al., in press). This dual effect may interfere with the endogenous (auto)antigen processing and loading to MHCII molecules and as a consequence, lead to the lower activation of autoreactive T cells that was previously shown experimentally (Monneaux et al., 2004; Monneaux et al., 2007).

Recent research suggests that autophagy is potentially increased in RA, as well as in other autoimmune diseases (Table 3; Wilhelm & Muller, submitted). This activation has been proposed for Crohn's disease (CD), RA, polymyositis (PM) and multiple sclerosis (MS), but not in autoimmune diabetes where, in contrast, autophagy might be decreased.

TABLE 3
List of autoimmune diseases with autophagy failures

| Autoimmune | Associated | | |
|---|---|---|---|
| diseases | genes | Cellular Dysfunctions | References |
| CD ⁽¹⁾ | ATG16L1 | | Hampe et al. 2007 |
| | IRGM | | Glas et al. 2003; |
| | | | Lu et al. 2013 |
| SLE | ATG5 | | Harley et al. 2008 |
| | | | Thou et al. 2011 |
| | DRAM1 | | Yang et al. 2013 |
| | PRDM1 | | Zhou et al. 2011 |
| | | MaA increased in T cells from MRL/lpr and NZB/W | Gros et al. 2012 |
| | | mice and from patients: autophagic vacuoles | |
| | | over-represented (WB, EM) ⁽²⁾ | |
| | | MaA deregulated in naïve CD4⁺T cells from patients: | Alessandri et al. 2012 |
| | | autophagosome-associated marker MAP1LC3 increased | |
| | | (WB) | |
| | | MaA hyper-activated in B cells from NZB/W mice and | Clarke et al. 2014 |
| | | naïve B cells of patients; autophagosomes number increased | |
| | | (FACS, FM) | |
| | | MaA activated in macrophages from lupus-prone mice and | Li et al. 21014 |
| | | patients: ATG5, ATG12 and BECN1 expression increased | |
| | | Increased HSPA8 expression in B and T cells of MRL/lpr | Page et al. 2011 |
| | | mice (WB, FACS, PCR) | |
| | | Increased LAMP-2A and CTSD expression in B cells of | Macri et al., in press |
| | | MRL/lpr mice; lysosomes are defective in MRL/lpr mice | |
| | | (WB, FACS, Q-PCR, in vitro assay for CMA) | |
| RA | ATG5 | | Orozco et al. 2011 |
| | ATG7 | | Lin et al. 2013 |
| | BECN1 | | Lin et al. 2013 |
| | | MaA activated in osteoclasts from patients: BECN1 and | Lin et al. 2013 |
| | | ATG7 expression increased (WB) | |
| | | Autophagic process increased in synovial fibroblast: | Kato et al. 2014 |
| | | p62 and MAP1LC3 expression increased (WB, FM) | |
| PM | | MaA activated in muscle fiber: MAP1LC3, CTSD and | Nogalska et al. 2010 |
| | | CTSB expression increased (WB) | |
| MS | ATG5 | | Mayes et al. 2014, |
| | | | Alirezaei et al. 2009 |
| | | MaA deregulated in T cells: ATG5 expression increased | Alirezaei et al. 2009 |
| | | (WB, PCR) | |
| Type 1 diabetes | | MaA diminished in diabetic mouse heart: MAP1LC3 and | Xu et al. 2013; |
| | | ATG5/12 expression reduced (WB, FM) | Yamahara et al. 2013 |

⁽¹⁾ Abbreviations: ATG, autophagy related-gene; BECN1, beclin-1; CD, Crohn’s disease; CMA, chaperone-mediated autophagy; CTSB, cathepsins B; CTSD, cathepsins D; DRAM1, damage-regulated autophagy modulator; EM, electron microscopy; FM, fluorescence microscopy; HSPA8, heat shock protein 8; IRGM, Immunity-related GTPase family M protein; LAMP-2A, lysosomal-associated membrane protein 2A; MaA, macroautophagy, MAP1LC3, microtubule-associated protein light chain 3; MS, multiple sclerosis; PCR, polymerase chain reaction; PM, polymyositis; PRDM1, positive regulatory domain 1-binding factor 1; RA, rheumatoid arthritis; SLE. systemic lupus erythematosus; WB, Western blot.
⁽²⁾ The method used to evaluate these changes is given in parentheses.

Ex vivo, P140 does not induce proliferation of peripheral T cells from lupus patients (in contrast to the non-phosphorylated form that does and in contrast to the data shown ex vivo in MRL/lpr context) but generates secretion of high levels of regulatory cytokine IL-10 in cell cultures (Monneaux et al., 2005). No proliferation and no IL-10 production were observed in the cultures when T cells from patients with other autoimmune diseases were tested (Monneaux et al., 2005). Patients (n=27) with rheumatoid arthritis (RA), primary Sjögren's syndrome, autoimmune deafness, polymyositis, primary billiary cirrhosis and autoimmune hepatitis were evaluated, as well as 4 patients hospitalized for non-autoimmune or infectious diseases.

These data (raised with small groups of patients) led us to conclude that most likely peptide P140 very specifically stimulates peripheral lupus CD4⁺ T cells but not T cells from patients with other pathophysiological conditions (Monneaux et al., 2005). These data were also against the potential effect of P140 peptide as a possible regulator of autophagy defects in these diseases.

Next, P140 peptide was administered in a model of mice that develop a RA-like disease (we anticipated to use this mouse model as a negative control of MRL/lpr-lupus prone mice). This model, called collagen-induced arthritis (CIA) mouse model, is the most commonly studied autoimmune model of RA. In this model, autoimmune arthritis is induced by immunizing DBA/1 mice with an emulsion of complete Freund's adjuvant (CFA) and type II collagen (CII), and typically, the first signs of arthritis appear in 21-28 days after immunization (Brand et al., 2007). CIA shares several pathological features with human RA, and CII is a major protein in cartilage, the target tissue of RA. Pathological features include synovial hyperplasia, mononuclear cell infiltration, and cartilage degradation. Susceptibility in these mice is linked to the expression of specific MHC class II genes, DBA/1 have H-2^{q} haplotype.

P140 peptide was thus administrated intravenously to DBA/1 mice at day −1, +7, +14 and +20 in a setting close to the one we used in MRL/lpr mice (100 μg/injection/mouse). CII in CFA was injected at days +1 and +21 (200 μg, intradermal route). Mouse weight and their clinical score were followed using very classical procedure. Biological parameters were also evaluated (i.e. T cell response, antibody response, joint histology, etc).

The results obtained in this experiment show that CD4⁺ T splenocytes from mice that receive the scrambled peptide ScP140 proliferate normally ex vivo in the presence of CII added to the cultures (FIG. 7; 100 μg CII/mL; measured using the CFSE assay by FACS). In sharp contrast, however, proliferation was strongly diminished when CD4 T cells were collected from the spleen of mice that receive P140 peptide (p=0.0539 between ScP140 and P140).

No effect was observable when CD8⁺ T cells were tested in the same conditions. Further results are awaited that will characterize this response in much more details. Histology will also complete these cellular data.

In any case, these results, which could not be anticipated, suggest an operational scheme that could mimic in RA the one found when we tested CD4⁺ T cells from P140-treated MRL/lpr lupus-prone mice. In MRL/lpr mice, P140 induces a significant decrease of MHCII expression at the B cells surface (via its effect on CMA), lowering therefore the presentation of antigenic peptide by antigen-presenting cells, which, as a matter of consequences, leads to a decreased reactivity of peripheral autoreactive T cells and improvement of disease condition. Thus, the data show that P140 peptides can be effective in a variety of other pathological conditions in which reduction of CMA activity would be desired.

Nowadays, there is no available data showing at the cellular level that CMA is altered in RA. No information exists regarding the properties of lysosomes in this pathology. Future investigation should be focused on the possible demonstration that autophagic flux is increased in mice with RA and B cells from RA patients, and CMA altered in this setting.

Other pathophysiological settings will be tested to accumulate pertinent data, notably in CD, PM, scleroderma (SSc) and MS. Established murine models are available for CD (e.g. IL-10 KO mice, SAMP1/YitFc mice, or the peptidoglycan-polysaccharide model using inbred rats) and MS (mouse and rat models of experimental autoimmune encephalomyelitis, EAE). Nowadays, however, good animal models do not exist for PM and SSc.

### Example 6: Endocytosis of P140 Particles

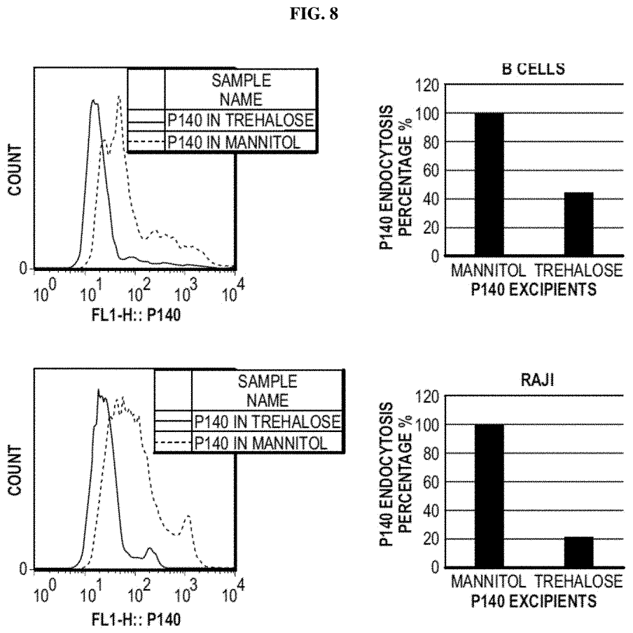

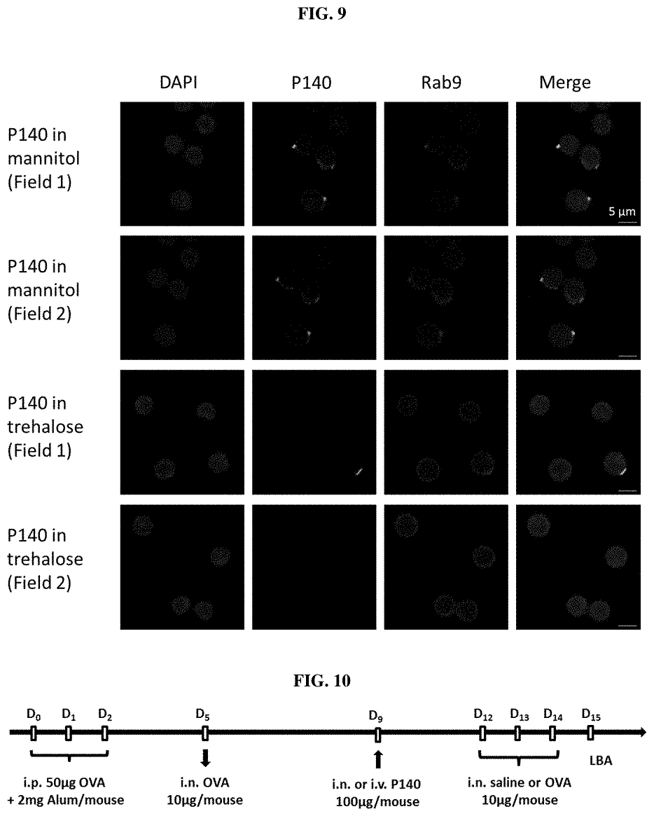

For P140 peptide activity, HSC70 binding and endocytosis appear to be important. It is believed that endocytosis must occur through the clathrin route. This implies that peptide+excipient should have a size in the range of 30 to 500 nm in diameter. For example P140+mannitol are in the 100 nm region whereas P140+trehalose are below 10 nm and therefore not effective binding to HSC70. For example, FIG. 8 shows cellular uptake of fluorescent P140 peptide in 5.4% mannitol or 10% trehalose in MRL/lpr B cells and Raji cells as visualized by flow cytometry. B cells were from 12-14 week-old MRL/lpr mice (primary cells); Raji cells are an established cell line derived in 1963 from B-lymphocyte of a patient with Burkitt's lymphoma. Much less cellular uptake of P140 in both MRL/lpr B cells and Raji cells when the peptide is diluted in trehalose than in mannitol. This result was confirmed using confocal microscopy (FIG. 9). The confocal images show the late endosomal compartment where P140 localizes before homing into lysosomes; DAPI identifies DNA. The results confirm the flow cytometry results that when in trehalose, P140 peptide enters B cells much less (See Tables 4 and 5).

TABLE 4
P140 on HSC70

| Peptide- | | | | | Conc | | | | | |
|---|---|---|---|---|---|---|---|---|---|---|
| concen- | ka | kd | Rmax | RI | of | KA | KD | Req | kobs | |
| tration | (1/Ms) | (1/s) | (RU) | (RU) | analyte | (1/M) | (M) | (RU) | (1/s) | Chi2 |
| | 450 | | 83.3 | | | | | | | 3.17 |
| P140- | | 3.12E− | | 12.1 | 1.56 u | 1.44E+ | 6.94E− | 15.3 | 3.82E− | |
| 1.56 μM | | 03 | | | | 05 | 6 | | 03 | |
| P140- | | 3.12E− | | 20.9 | 3.12 u | 1.44E+ | 6.94E− | 25.8 | 4.52E− | |
| 3.12 μM | | 03 | | | | 05 | 6 | | 03 | |
| P140- | | 3.12E− | | 33.8 | 6.25 u | 1.44E+ | 6.94E− | 39.5 | 5.93E− | |
| 6.25 μM | | 03 | | | | 05 | 6 | | 03 | |
| P140- | | 3.12E− | | 62.5 | 12.5 u | 1.44E+ | 6.94E− | 53.6 | 8.74E− | |
| 12.5 μM | | 03 | | | | 05 | 6 | | 03 | |
| P140- | | 3.12E− | | 118 | 25 u | 1.44E+ | 6.94E− | 65.2 | 0.0144 | |
| 25 μM | | 03 | | | | 05 | 6 | | | |

TABLE 5
P140 (MO) on HSC70

| Peptide- | | | | | Conc | | | | | |
|---|---|---|---|---|---|---|---|---|---|---|
| concen- | ka | kd | Rmax | RI | of | KA | KD | Req | kobs | |
| tration | (1/Ms) | (1/s) | (RU) | (RU) | analyte | (1/M) | (M) | (RU) | (1/s) | Chi2 |
| | 1.15E+3 | | 39 | | | | | | | 1.18 |
| P140 | | 2.20E− | | 14 | 1.56 u | 5.24E+ | 1.91E− | 17.6 | 4.00E− | |
| (MO)- | | 3 | | | | 5 | 6 | | 03 | |
| 1.56 μM | | | | | | | | | | |
| P140 | | 2.20E− | | 18.7 | 3.12 u | 5.24E+ | 1.91E− | 24.2 | 5.80E− | |
| (MO)- | | 3 | | | | 5 | 6 | | 03 | |
| 3.12 μM | | | | | | | | | | |
| P140 | | 2.20E− | | 25.9 | 6.25 u | 5.24E+ | 1.91E− | 29.9 | 9.40E− | |
| (MO)- | | 3 | | | | 5 | 6 | | 03 | |
| 6.25 μM | | | | | | | | | | |
| P140 | | 2.20E− | | 36.9 | 12.5 u | 5.24E+ | 1.91E− | 33.9 | 0.0166 | |
| (MO)- | | 3 | | | | 5 | 6 | | | |
| 12.5 μM | | | | | | | | | | |
| P140 | | 2.20E− | | 53.4 | 25 u | 5.24E+ | 1.91E− | 36.3 | 0.031 | |
| (MO)- | | 3 | | | | 5 | 6 | | | |
| 25 μM | | | | | | | | | | |

### Example 7. Anti-Inflammatory Effect of the P140 Phosphopeptide in a 15-Day Model of Eosinophilic Airway Inflammation Induced by Ovalbumin in Mice

The anti-inflammatory effect of the P140 phosphopeptide was evaluated when administered locally (intranasally) or systemically (intravenously) in a 15-day model of hypereosinophilic airway inflammation in mice.

The P140 phosphopeptide was solubilized in sterile water (Braun) and 10× concentrate sterile saline was added to adjust osmolarity to 300 mosm. Osmolarity was controlled with a micro osmometer (Löser, type 15) and validated (302 mosm).

The P140 phosphopeptide was used in vivo at the dose of 4 mg/kg by intranasal (i.n.) and intravenous (i.v.) routes. Control animals received equivalent volumes (1 ml/kg for i.n. and 2 ml/kg for i.v.) of saline (Table 6).

Nine-week-old male Balb/c mice were sensitized by intraperitoneal (i.p.) injections of a mixture containing 50 μg OVA (Sigma-Aldrich) and 2 mg alum (Sigma-Aldrich) in 0.1 ml saline. Mice were challenged by i.n. administration of 25 μl of OVA on day 5, then 25 μl of OVA and/or saline on day 12, 13 and 14. Mice were treated by i.v. injection (2 ml/kg) or i.n. administration (1 ml/kg) of P140 or solvent on day 9 (See FIG. 9).

TABLE 6

| Group | Number | | |
|---|---|---|---|
| Number | of Mice | Treatment | Challenge |
| 1 | 1 | Solvent | Saline |
| 2 | 2 | P140 (i.n.) | Saline |
| 3 | 2 | P140 (i.v.) | Saline |
| 4 | 5 | Solvent | OVA |
| 5 | 6 | P140 (i.n.) | OVA |
| 6 | 6 | P140 (i.v.) | OVA |

BAL was performed twenty-four hours after LPS challenge as described (Daubeuf, F. and Frossard, N. 2012. Performing Bronchoalveolar Lavage in the Mouse. Curr Protoc Mouse Biol 2:167-175). Mice were anaesthetized IP (Ketamine 150 mg/kg—Xylasine 10 mg/kg). Blood was collected from the heart, centrifuged at 10,000 g for 2 min and serum stored at −20° C. After semi-excision of the trachea, a plastic cannula was inserted, and airspace washed with 0.5 ml of 0.9% NaCl injected with a 1 ml syringe. This procedure was performed 10 times. The initial concentrated supernatant of the 2 first lavages (volume=2×0.5 ml administered, ˜0.5 ml recovered) was collected for cytokine measurements. The remaining BAL fluid was centrifuged (300 g for 5 min, 4° C.), and cell pellets pooled. The cell pellet was suspended in 500 μl of 0.9% NaCl and used for total cell counts evaluated on a Muse® Cell Analyser. Differential cell counts were assessed by flow cytometry (LSRII® cytometer, BD Bioscience). BAL cells were added with FCblock (0.5 μl, 553142, BD Bioscience) in a black microplate, incubated for 20 min at room temperature. Then, marker antibodies were added: CD11c-FITC (557400, BD bioscience), Gr-1-Pe-eFluor610 (61-5931-82, eBioscience), CD11b-APC-Cy7 (557657, BD bioscience), CD45-AlexaFluor700 (103128, BioLegend), CD3-BV605 (564009, BD bioscience), CD19-PE-Cy7 (552854, BD bioscience). Antibodies were incubated with BAL cells for 30 min at room temperature before DAPI (5 μl, BD bioscience) addition, and flow cytometry was performed immediately.

Data are presented as means±SEM. Differences between groups were tested for statistical significance using one-way ANOVA followed by Tukey's post-test. For statistical analysis, control groups 1, 2 and 3 were pooled. Data were considered significantly different when p≤0.05.

Analysis of airway cells recovered in BAL fluid in control mice challenged with saline shows that the P140 phosphopeptide administered i.n. or i.v. has little effect per se on the number of cells recovered in BAL fluid as compared to vehicle (saline), and in particular has no pro-inflammatory effect. (See Table 7).

TABLE 7

| | Mice | Total cells | Macrophages | Eosinophils | Neutrophils | T cells | B cells |
|---|---|---|---|---|---|---|---|
| Ctrl | NL415-2_1 | 333568 | 328362 | 149 | 223 | 4834 | 149 |
| P140-IN | NL415-2_2 | 392461 | 388102 | 168 | 56 | 4135 | 112 |
| P140-IN | NL415-2_8 | 438573 | 434029 | 103 | 61 | 4180 | 242 |
| P140-IV | NL415-2_4 | 341738 | 335658 | 110 | 259 | 5311 | 70 |
| P140-IV | NL415-2_15 | 340389 | 335200 | 166 | 133 | 4790 | 266 |
| OVA | NL415-2_3 | 1658393 | 563095 | 888525 | 78637 | 128136 | 21766 |
| OVA | NL415-2_5 | 1098900 | 331150 | 626131 | 45797 | 95822 | 25365 |
| OVA | NL415-2_9 | 1546822 | 388693 | 1022052 | 68833 | 67243 | 25216 |
| OVA | NL415-2_14 | 1468429 | 418191 | 833452 | 95942 | 120843 | 15380 |
| OVA | NL415-2_19 | 1064136 | 302118 | 624692 | 80691 | 56635 | 24624 |
| P140-IN | NL415-2_6 | 862995 | 271110 | 490306 | 57542 | 44036 | 25606 |
| P140-IN | NL415-2_7 | 942875 | 322340 | 497948 | 60787 | 61800 | 32251 |
| P140-IN | NL415-2_10 | 1120576 | 247391 | 737671 | 62354 | 73159 | 26562 |
| P140-IN | NL415-2_11 | 1592328 | 538954 | 839841 | 95173 | 118360 | 23383 |
| P140-IN | NL415-2_16 | 1377755 | 436210 | 792249 | 47346 | 101951 | 33156 |
| P140-IN | NL415-2_20 | 1028339 | 286509 | 615171 | 65366 | 61293 | 13236 |
| P140-IV | NL415-2_12 | 949720 | 439265 | 425928 | 42783 | 41744 | 10219 |
| P140-IV | NL415-2_13 | 780142 | 442055 | 272763 | 21442 | 43881 | 15209 |
| P140-IV | NL415-2_17 | 809921 | 244523 | 473105 | 59616 | 32677 | 14027 |
| P140-IV | NL415-2_18 | 895467 | 293070 | 470027 | 76867 | 55502 | 17417 |
| P140-IV | NL415-2_21 | 738452 | 342134 | 327186 | 40275 | 28857 | 11003 |
| P140-IV | NL415-2_22 | 885821 | 379565 | 429469 | 31756 | 45030 | 10922 |

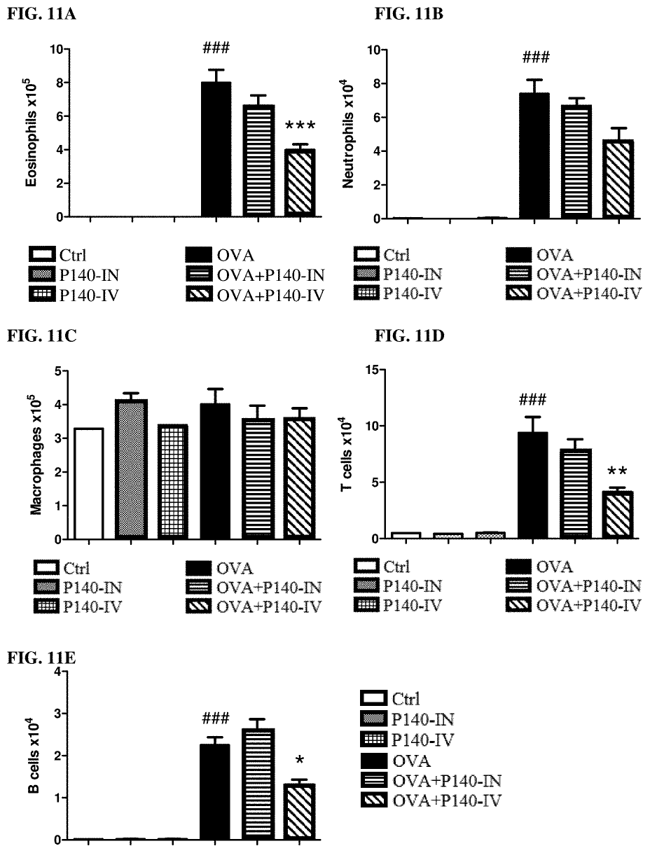

In ovalbumin-challenged mice, the total number of inflammatory cells recovered in BAL fluid increases significantly. This effect is related to significant increased influx of eosinophils, neutrophils, T and B cells (###p<0.001; FIG. 11A-11E).

The P140 phosphopeptide administered i.v. (4 mg/kg) significantly decreases eosinophil (−50%, ***p<0.001), T cells (−66%, **p<0.01) and B cells (−42%, *p<0.05) recruitment, as well as neutrophils recruitment (−38%) although not below the significance cutoff. By contrast, administered locally by i.n. route, the P140 phosphopeptide shows little effect on inflammatory cell recruitment in BAL, suggesting P140 is acting through a systemic effect.

The project aimed at studying whether the P140 phosphopeptide could have an anti-inflammatory effect administered locally by i.n. or systemically by i.v. in a 15-day airway hypereosinophilia model in Balb/c mice sensitized and challenged with ovalbumin. We compared the effect of P140 administered i.n. or i.v. 2 days before OVA or saline challenge, i.e. 6 days before airway inflammatory cell recovery by bronchoalveolar lavage.

Thus, i.v. administration (4 mg/kg) of P140 shows anti-inflammatory effect in this airway hypereosinophilia model to OVA in Balb/c mice, whereas i.n. administration remains without substantial effect. This suggests the anti-inflammatory activity of P140 is a systemic (e.g., spleen, lymphoid organs, bone marrow) rather than a local effect.

### Example 8. Study of the P140 Peptide Effect in a Mouse Model of Colonic Inflammation (DSS-Induced Model)

Normal mice (C57BL/6; 7 week-old; males) have received the P140 peptide (100/injection, iv route; 10 mice) or saline only (control group; 10 mice) at days −2 and −1. At day 0, dextran sodium sulfate (DSS; 2-3%) was administrated to induce the disease.

Animals were examined every day for body weight loss, stool consistency, diarrhea, and blood in the feces. The animals were sacrificed around day 14 or at any time if they are very sick (loss >25% body weight). Statistics: Mann-Whitney (exact)

Little difference in the DAI (p=0.5386). However, this clinical index is not very well adapted to mouse model. There was a significant increase of the colon size, reflecting a decrease of inflammation (p=0.0011). No difference of the body weight was observed between the two groups. However there was a tendency at day +3 and day +4. The blood appeared in the feces at day +6 in the control groups versus day +8 only in the P140 group

### Example 9. Effect of the P140 Phosphopeptide in a 31-Day Model of Eosinophilic Airway Inflammation Induced by House Dust Mite Extract (HDM) in Mice

The aim of this study was to evaluate the effect of the P140 phosphopeptide administered systemically (intravenously) in a 31-day model of HDM-induced asthma in mice. The P140 phosphopeptide was solubilized in sterile water (Braun) and 10x concentrate sterile saline was added to adjust osmolarity to 300 mosm. Osmolarity was controlled with a micro osmometer (Löser, type 15) and validated (303 mosm). The P140 phosphopeptide was used in vivo at the dose of 4 mg/kg by intravenous (i.v.) routes. Control animals received equivalent volumes (2 ml/kg) of saline (Table 8).

TABLE 8

| Group | Number | | Challenge |
|---|---|---|---|
| Number | of Mice | Treatment | (D₃₈-D₃₀) |
| 1 | 6 | Solvent | Saline |
| 2 | 5 | P140 (i.v.) 4 mg/kg | Saline |
| 3 | 8 | Solvent | HDM |
| 4 | 8 | P140 (i.v.) 4 mg/kg | HDM |

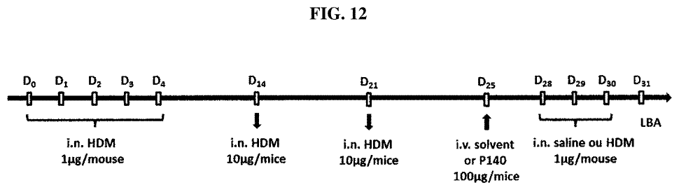

Nine-week-old male Balb/c mice were sensitized by intranasal (i.n.) administration of HDM extract (Stallergenes): 1 μg in 25 μl saline on days 0, 1, 2, 3, 4, and 10 μg on days 14 and 21. Mice were challenged by i.n. administration of HDM (1 μg) and/or saline on days 28, 29 and 30. Mice were treated by i.v. injection (2 ml/kg) of P140 or solvent on day 25 (see FIG. 12).

Airway response to Methacholine (Flexivent®). On day 31, airway responses to PBS then methacholine were assessed using a forced oscillation technique (Flexivent®, SCIREQ, Montreal, Canada) as described (Daubeuf et al, Bioprotocol, 645, 2013). Mice were anesthetized with an intraperitoneal injection of xylasine (Rompun®; 1 mg/kg), followed fifteen minutes later by an intraperitoneal injection of pentobarbital sodium (3.64 mg/Kg). The trachea was exposed and an 18-gauge metal needle was inserted into the trachea. Airways were connected to a computer-controlled small animal ventilator, and quasi-sinusoidally ventilated with a tidal volume of 10 ml/Kg at a frequency of 150 breaths/min and a positive end-expiratory pressure of 2 cm H2O to achieve a mean respiratory volume close to that of spontaneous breathing. After baseline measurement, each mouse was challenged for 10 sec with an aerosol of PBS generated with an in-line nebulizer and administered directly through the ventilator. Then, aerosolized methacholine (MCh) at 50 mg/ml was administered for 10 sec. The effect of methacholine was calculated as the peak response, i.e. the mean of the three maximal values integrated for calculation of airway resistance (R, cm H₂0·s·mL⁻¹), elastance (E, cm H₂0·mL⁻¹) and compliance (C, mL·cm H₂0⁻¹).

BAL was performed after airway responsiveness measurement twenty-four hours after HDM challenge as described (Daubeuf et al. 2012). Mice were anaesthetized IP (Ketamine 150 mg/kg—Xylasine 10 mg/kg). Blood was collected from the heart, centrifuged at 10,000 g for 2 min and serum stored at −20° C.

After semi-excision of the trachea, a plastic cannula was inserted, and airspace washed with 0.5 ml of 0.9% NaCl injected with a 1 ml syringe. This procedure was performed 10 times. The initial concentrated supernatant of the 2 first lavages (volume=2×0.5 ml administered, approximately 0.5 ml recovered) was collected for cytokine measurements. The remaining BAL fluid was centrifuged (300 g for 5 min, 4° C.), and cell pellets pooled. The cell pellet was suspended in 500 μl of 0.9% NaCl and used for total cell counts evaluated on a Muse® Cell Analyser (Millipore). Differential cell counts were assessed by flow cytometry (LSRII® cytometer, BD Bioscience). BAL cells were added with FCblock (0.5 μl, 553142, BD Bioscience) in a black microplate, incubated for 20 min at room temperature. Then, marker antibodies were added: CD11c-FITC (557400, BD bioscience), Gr-1-PeeFluor610 (61-5931-82, eBioscience), F4/80-PE (12-4801-82, eBioscience), CD11b-APC-Cy7 (557657, BD bioscience), CD45-AlexaFluor700 (103128, BioLegend), CD3-BV605 (564009, BD bioscience), CD19-PE-Cy7 (552854, BD bioscience). Antibodies were incubated with BAL cells for 30 min at room temperature before DAPI (5 μl, BD bioscience) addition, and flow cytometry was performed immediately.

All mice were sensitized to HDM on days 0, 1, 2, 3, 4, 14, 21, and challenged either with saline (chronic asthma) or HDM (challenge with allergen). Results are presented as means±SEM. Differences between groups were tested for statistical significance using Student's t test for inflammatory cells and a two-way ANOVA followed by Bonferroni post-test for airway responses. Data were considered significantly different when p≤0.05.

TABLE 9

| | Mice | Total cells | Macrophages | Eosinophils | Neutrophils | T cells | B cells | DCs |
|---|---|---|---|---|---|---|---|---|
| NL715-3 | Ctrl | 979000 | 189476 | 355097 | 20526 | 207329 | 17702 | 454 |
| NL715-26 | Ctrl | 767000 | 25268 | 403090 | 12409 | 267950 | 33315 | 301 |
| NL715-30 | Ctrl | 386000 | 25101 | 205264 | 3229 | 109936 | 17394 | 156 |
| NL715-33 | Ctrl | 768000 | 7433 | 443543 | 96949 | 178871 | 34094 | 0 |
| NL715-35 | Ctrl | 913000 | 9396 | 500534 | 139632 | 202210 | 51609 | 270 |
| NL715-37 | Ctrl | 801000 | 12384 | 384299 | 145765 | 222421 | 23747 | 222 |
| NL715-1 | P140 | 448000 | 130734 | 124514 | 2584 | 57711 | 2297 | 0 |
| NL715-5 | P140 | 1390000 | 267685 | 450346 | 35734 | 323523 | 44688 | 484 |
| NL715-8 | P140 | 1510000 | 360987 | 448074 | 23986 | 294011 | 22879 | 461 |
| NL715-11 | P140 | 815000 | 177815 | 205568 | 34836 | 208439 | 11101 | 319 |
| NL715-25 | P140 | 484000 | 73725 | 239527 | 2394 | 86810 | 7660 | 160 |
| NL715-4 | HDM | 2210000 | 90204 | 1054534 | 438909 | 507196 | 29645 | 231 |
| NL715-6 | HDM | 1810000 | 63322 | 842282 | 396394 | 372799 | 72101 | 329 |
| NL715-9 | HDM | 2330000 | 73284 | 1312253 | 365962 | 444121 | 62314 | 457 |
| NL715-12 | HDM | 2190000 | 118970 | 976204 | 344747 | 543880 | 87229 | 1390 |
| NL715-14 | HDM | 3077000 | 89870 | 1466110 | 663136 | 573814 | 193834 | 915 |
| NL715-28 | HDM | 1470000 | 70438 | 561204 | 379937 | 355637 | 32838 | 493 |
| NL715-36 | HDM | 3500000 | 185702 | 1850328 | 73656 | 1076717 | 124546 | 4018 |
| NL715-38 | HDM | 2430000 | 94477 | 1325575 | 33515 | 776133 | 106880 | 1056 |
| NL715-2 | HDM + P140 | 2140000 | 58705 | 955824 | 606700 | 400329 | 59092 | 1034 |
| NL715-7 | HDM + P140 | 2992000 | 118771 | 1404942 | 510027 | 735865 | 102246 | 2238 |
| NL715-10 | HDM + P140 | 2190000 | 314326 | 636065 | 500004 | 383074 | 42085 | 1793 |
| NL715-13 | HDM + P140 | 1010000 | 126002 | 342046 | 147616 | 243813 | 25443 | 242 |
| NL715-27 | HDM + P140 | 2310000 | 34364 | 1283190 | 371317 | 469834 | 116814 | 586 |
| NL715-29 | HDM + P140 | 2220000 | 38709 | 1036803 | 487119 | 502985 | 115000 | 1350 |
| NL715-31 | HDM + P140 | 1270000 | 29121 | 538210 | 284503 | 334784 | 53947 | 733 |
| NL715-34 | HDM + P140 | 2410000 | 73491 | 1010686 | 24426 | 1056303 | 170980 | 1928 |

| | | Rrs | | Crs | | Ers | |
|---|---|---|---|---|---|---|---|
| Mice | | cmH2O.s/mL | | mL/cmH2O | | cmH2O/mL | |
| NL715-3 | Ctrl | 0.7599 | 4.7857 | 0.0586 | 0.0270 | 17.0739 | 38.9111 |
| NL715-30 | Ctrl | 0.4768 | 4.5462 | 0.0598 | 0.0172 | 16.7201 | 62.7641 |
| NL715-33 | Ctrl | 0.8317 | 8.8241 | 0.0466 | 0.0079 | 21.5098 | 147.5663 |
| NL715-35 | Ctrl | 0.5620 | 9.2466 | 0.0536 | 0.0053 | 18.6679 | 233.9273 |
| NL715-37 | Ctrl | 0.6316 | 11.2979 | 0.0501 | 0.0083 | 19.9557 | 143.6106 |
| NL715-1 | P140 | 0.5590 | 4.1067 | 0.0545 | 0.0304 | 18.3671 | 33.3720 |
| NL715-5 | P140 | 0.8945 | 9.4002 | 0.0498 | 0.0132 | 20.0811 | 84.9688 |
| NL715-8 | P140 | 0.5926 | 2.2229 | 0.0569 | 0.0380 | 17.5777 | 26.3200 |
| NL715-11 | P140 | 0.8074 | 4.0926 | 0.0541 | 0.0238 | 18.4893 | 42.4174 |
| NL715-25 | P140 | 0.4418 | 2.1844 | 0.0650 | 0.0370 | 15.3975 | 28.6047 |
| NL715-4 | HDM | 1.0205 | 9.0924 | 0.0537 | 0.0081 | 18.6618 | 128.4742 |
| NL715-6 | HDM | 0.9134 | 5.3264 | 0.0452 | 0.0099 | 21.0856 | 173.5847 |
| NL715-9 | HDM | 0.5742 | 6.4096 | 0.0537 | 0.0141 | 18.6092 | 104.8681 |
| NL715-12 | HDM | 0.8239 | 9.3617 | 0.0528 | 0.0056 | 18.9503 | 224.6403 |
| NL715-14 | HDM | 0.6807 | 7.4437 | 0.0493 | 0.0103 | 20.2677 | 156.8403 |
| NL715-28 | HDM | 0.6958 | 5.0333 | 0.0533 | 0.0126 | 18.7594 | 87.2749 |
| NL715-36 | HDM | 0.9430 | 14.8440 | 0.0573 | 0.0051 | 17.5010 | 214.1691 |
| NL715-38 | HDM | 0.7308 | 8.7652 | 0.0538 | 0.0152 | 18.5827 | 71.7470 |
| NL715-2 | HDM + P140 | 0.6405 | 5.6421 | 0.0554 | 0.0146 | 18.0582 | 79.8018 |
| NL715-7 | HDM + P140 | 0.6092 | 10.3886 | 0.0514 | 0.0075 | 19.4433 | 148.4985 |
| NL715-10 | HDM + P140 | 0.7972 | 11.9654 | 0.0528 | 0.0062 | 18.9515 | 188.1151 |
| NL715-13 | HDM + P140 | 0.5185 | 10.5419 | 0.0566 | 0.0065 | 17.6781 | 219.9720 |
| NL715-27 | HDM + P140 | 0.6804 | 8.8810 | 0.0492 | 0.0101 | 20.3326 | 125.7688 |
| NL715-29 | HDM + P140 | 0.6365 | 13.0087 | 0.0458 | 0.0060 | 21.8367 | 173.4582 |
| NL715-31 | HDM + P140 | 0.4744 | 7.0705 | 0.0552 | 0.0138 | 18.1106 | 72.6033 |
| NL715-34 | HDM + P140 | 0.5456 | 9.7688 | 0.0589 | 0.0081 | 16.9723 | 131.8035 |

Airway Responses in Chronic Asthma

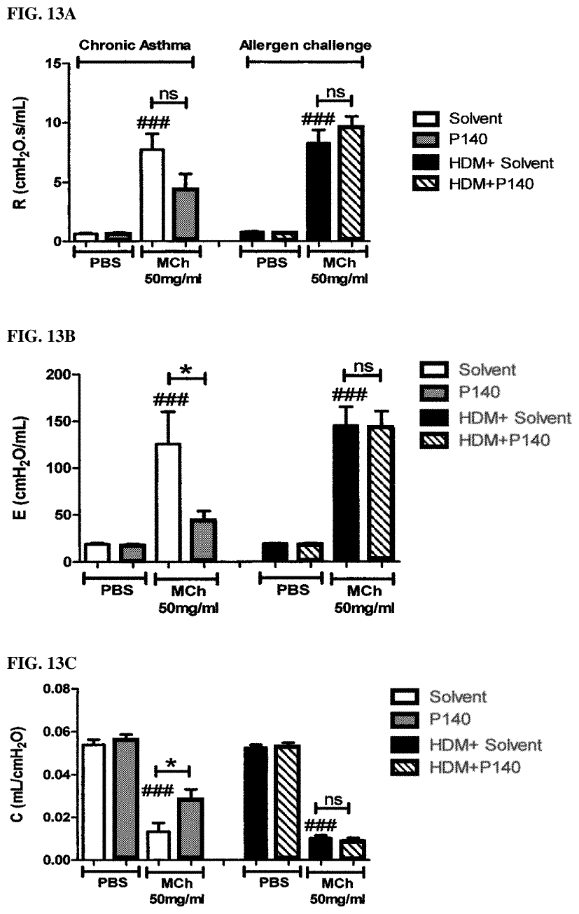

Inhalation of PBS had no effect on baseline airway resistance, elastance and compliance assessed by the Flexivent® technique in saline-challenged, solvent-treated mice (FIG. 13A-C). Treatment with P140 (i.v., 4 mg/kg, day 25) also had no effect on any parameter as compared to solvent-treated mice (FIG. 13A-C). However, inhalation of methacholine (50 mg/ml) induced a marked increase in airway resistance and elastance accompanied with a decrease in compliance (FIGS. 13A, B and C, respectively) in saline-challenged, solvent-treated mice. Treatment with P140 significantly decreased elastance (−65%, *p<0.05) and increased airway compliance (+115%, *p<0.05) as compared to the solvent group (FIG. 13B), as well as decreased airway resistance (−42%) although non-significantly (n=5).

Airway Responses in Mice Challenged with Allergen (HDM)

Inhalation of PBS had no effect on baseline airway resistance, elastance and compliance in HDM-challenged solvent-treated mice. Treatment with P140 had no effect on airway resistance, elastance or compliance in allergen-challenged mice as compared to the solvent group. However, inhalation of methacholine induced significant increases in airway resistance and elastance accompanied with a decrease in compliance in HDM-challenged, solvent-treated mice (FIGS. 13A and 13B).

Effect in Chronic Asthma (HDM-Sensitized, Saline-Challenged Mice)

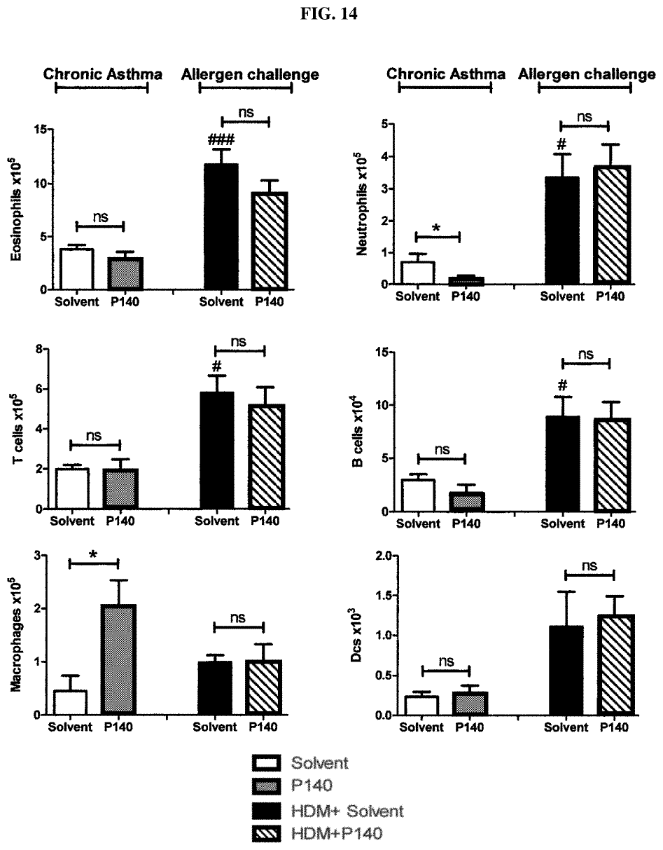

Eosinophils (3.8×10⁵), neutrophils (0.7×10⁵), macrophages (0.4×10⁵), T and B lymphocytes (1.9×10⁵ and 0.3×10⁵), and dendritic cells (0.2×10³) were recovered in BAL fluid upon saline challenge in solvent-treated mice (FIG. 14). Treatment with P140 (4 mg/kg i.v., day 25) significantly decreased the number of neutrophils (−71%, *p<0.05), as well as eosinophils (−25%) and B cells (−40%) although non-significantly, and significantly increased the number of macrophages by 4.5-fold (*p<0.05) as compared to the solvent group (FIG. 14).

Effect in Mice Challenged with Allergen (HDM-Sensitized and HDM-Challenged)

The number of inflammatory cells recovered in BAL fluid in HDM-challenged mice significantly increased as compared to chronic asthma (saline-challenged) (FIG. 14). This effect was related to a significant increased influx of eosinophils (11.7×105, ###p<0.001), neutrophils (3.4, #p<0.05), T and B cells (5.8×105 and 0.9×105, #p<0.05) (FIG. 14) in response to HDM challenge. Thus, treatment with P140 showed no effect on the inflammatory cell recruitment in BAL in HDM-challenged mice in comparison to the solvent group.

The aim of this study was to evaluate whether the P140 phosphopeptide could have an antiasthmatic effect when administered systemically in a 31-day asthma model in Balb/c mice sensitized to house dust mite (HDM) extracts. P140 was administered i.v. in HDM-sensitized mice, 2 days before HDM or saline challenge, i.e. 6 days before assessment of airway responses to MCh and of airway inflammatory cell recovery in the bronchoalveolar lavage.

We chose to design the study as sensitizing all animals to HDM as i) a model of chronic asthma when animals were further challenged with saline (HDM-sensitized, saline-challenged mice), and ii) a model of allergen challenge-induced asthma attack, when animals were further challenged with HDM (HDM-sensitized, HDM-challenged mice). In that, the protocol design could show the effect of P140 i) in every day chronic asthma, as well as ii) during asthma crisis.

In mice with chronic asthma (HDM-sensitized and saline-challenged) Methacholine induced a large increase in airway obstruction measured as increases in airway resistance (R) and elastance (E), accompanied by a decrease in airway compliance (C). As compared to the normal values we use to observe for control, unsensitized and non-challenged Balb/c mice (baseline R, E and C), these values are representative of the presence of airway hyperresponsiveness in these mice with chronic asthma. We show that P140 treatment significantly decreased airway responses to MCh with significant decrease in airway elastance E and increase in compliance C, as well as decrease in airway resistance R although non-significant as compared to the solvent-treated group. This suggests P140 decreases airway hyperresponsiveness observed in our allergic chronic asthma model.

In addition, we observe in this study the effect of P140 treatment on the inflammatory reaction existing in the airways in chronic asthma. Our model of chronic asthma is characterized by infiltration of eosinophils, neutrophils, macrophages, dendritic cells, T and B cells. P140 treatment induced a significant decrease in the number of neutrophils recovered in the bronchoalveolar lavage, as well as of eosinophils and B cells although non-significantly, and a significant increase in macrophages, as compared to solvent-treated mice. Asthma is known as an eosinophilic inflammation of the airways. More importantly, difficult uncontrolled asthma is described as an airway inflammatory disease with a change in the infiltrated inflammatory cell phenotype, most importantly with neutrophils infiltrating the airways. This phenotype is often resistant to glucocorticoid treatment. Therefore, the effect observed with the P140 phosphopeptide suggests that P140 has an antiasthmatic potential in chronic asthma, on airway hyperresponsiveness as well as airway inflammation.

Without being bound by any particular theory, P140 appears to be enhancing the resolution of chronic inflammation, in particular for neutrophils, existing in the airways in asthma, accompanied with resolution of airway hyperresponsiveness, which is one of the most invalidating symptom in asthma patients. In mice challenged with allergen (HDM-sensitized and HDM-challenged) HDM induced further increase in airway hyperresponsiveness and airway inflammatory cell infiltrate recovered in BAL. However, P140 treatment had little effect on this allergen-challenge-induced increased airway hyperresponsiveness to MCh nor inflammatory cell recruitment in BAL. This indicates that P140 treatment, when administered 2 days before allergen challenge is not as potent for blocking the reaction of an asthma crisis, although the basal levels of asthmatic airway responsiveness and inflammation in the absence of HDM challenge were reduced.

Systemic administration of P140 (4 mg/kg i.v.), 2 days before saline challenge, has the potential to restore baseline airway responsiveness, and resolve inflammation in every day chronic asthma. By contrast, in the conditions used for P140 administration, i.e. 2 days before the HDM challenge, P140 had no effect on the consequences of allergen challenge, indicating it does not improve nor worsen the effect of allergen in the sensitized airways. Such activity of P140 measured in the 31-day model of asthma indicates that P140 could be effective in chronic asthma. Increasing delay between P140 treatment(s) and allergen challenge might allow increased activity of P140 in asthma. We anticipate P140 may prevent airway hyperresponsiveness as well as airway inflammation caused by repeated allergen contact, i.e. resolve symptoms of every day chronic asthma.

### Example 10. Effect of p140 Peptide on a Rat Model for Chronic Inflammatory Demyelinating Polyradiculoneuropathy

Chronic inflammatory demyelinating polyradiculoneuropathy (CIDP) is an autoimmune-mediated inflammatory disease of the peripheral nervous system (PNS) for which therapies are limited/lacking. Recently, a new animal model for CIDP, the chronic-EAN, has been characterized (Brun S, Beaino W, Kremer L, Taleb O, Mensah-Nyagan A G, Lam C D, Greer J M, De Seze J, and Trifilieff T (2015). Characterization a new rat model for chronic inflammatory demyelinating polyradiculoneuropathies. J. Neuroimmunol. 278: 1-10). This model fulfills electrophysiological criteria of demyelination with axonal degeneration, confirmed by immunohistopathology. The late phase of the chronic disease was characterized by accumulation of IL-17 cytokine-positive cells and macrophages in sciatic nerves, and by high serum IL-17 levels. It is a reliable and reproducible animal model for CIDP, which can be used for translational drug studies for chronic human autoimmune-mediated inflammatory diseases of the PNS, and particularly CIDP, for which, there is a crucial need for new targeted immunotherapies. Thus, this study sought to investigate the possible effect of P140 peptide in this new preclinical rat model for CIDP.

Male Lewis rats, 7-8 weeks old, weighing 250-270 g, purchased from Charles River (Domaine des Oncins, L′Arbresle, France) were used. To induce chronic-EAN (CIDP), rats were immunized with S-palm-P0(180-199) peptide by subcutaneous injection at the base of the tail of 200 μL of an inoculum containing 200 μg of peptide (Ac(palm)KRGRQTPVLYAMLDHSRS), and 0.5 mg of Mycobacterium tuberculosis (strain H37 RA, Difco, Detroit, Mich., USA) emulsified in 100 μL of saline solution and 100 μL of Freund's incomplete adjuvant (SIGMA-Aldrich, St-Quentin Fallavier, France).

Body weight and clinical scores are assessed daily until 60 days post-immunization (dpi). Severity of paresis is graded as follows: 0=no illness; 1=flaccid tail; 2=moderate paraparesis; 3=severe paraparesis; 4=tetraparesis; 5=death.

A total of 15 rats were used and treated as indicated in the following table:

TABLE 10

| Number | | | |
|---|---|---|---|
| of rats | Emulsion injected at day 0 | Denomination | Treatment |
| 4 | S-palm P0(180-499) + CFA | control CIDP | — |
| 7 | S-palm P0(180-199) + CFA | treated CIDP | P140 |

100 μg/rat P140 peptide in 500 μL water/saline (1:10) were intraperitoneally injected at 5, 7, 9, 13 dpi and 3 times per week from 22 dpi until the end of the study.

a) Cytokine ELISA

Sera from treated and non-treated rats will be collected at 18, 40 and 60 dpi. The concentration of IL-17 cytokine will be measured in duplicate in undiluted sera using commercial ELISA kits specific for rat IL-17 (eBioscience, San Diego, Calif., USA), as per the manufacturers' instructions.

b) Antibody ELISA

Sera from treated and non-treated rats will also be tested at 18, 40 and 60 dpi for the presence of anti-P0(180-199) antibodies using ELISA. Peptide will be coated onto 96-well plates at 20 μg/mL in 0.05 M carbonate-bicarbonate buffer solution (pH 9.6, 100 μL/well) and incubated overnight at 4° C. Plates will be then washed with phosphate-buffered saline (PBS) and blocked with 1% bovine serum albumin in PBS for 1 h at 37° C. After washing, sera (100 μL/well) diluted at 1/5000 will be added in duplicate and incubated for 2 h at 37° C. After washing, plates will be incubated with goat anti-rat IgG coupled to peroxidase (1:2000, SIGMA-Aldrich) for 2 h at 37° C. After extensive washing, each well will be incubated with 75 μL of TMB at room temperature until color development. The reaction will be stopped by addition of 1 M H₂SO₄ (25 μL/well).

c) Immunohistochemistry

To evaluate inflammatory cell infiltration and pathological changes in the PNS, treated and non-treated rats will be sacrificed at 60 dpi. Rats will be deeply anesthetized with Ketamine/Rompun and perfused intracardially with 4° C., 4% (v/v) paraformaldehyde (PFA) in PBS. Sciatic nerves and cauda equina will be dissected out, fixed in Bouin and embedded in paraffin.

After dewaxing, cross-sections (5 μm) will be heated at 80° C. for 10 min in citrate buffer. Endogenous peroxidase will be inhibited with 0.02% H₂O₂ in water for 10 min. Non-specific binding sites will be blocked with 5% fetal calf serum (Gibco Invitrogen, Camarillo, Calif., USA) in PBS for 30 min and then with the following monoclonal antibodies: anti-MBP (1:500; produced in house) for myelin; SMI-311 (1:1000; Abcam, Paris, France) for neurofilaments; ED1 (1:400; Serotec, Oxford, UK) for macrophages and anti-interleukin-17 (IL-17; 1:100; Santa Cruz Biotechnology, Santa Cruz, Calif., USA). Antibody binding to tissue sections will be visualized with biotinylated anti-mouse IgG (1:200; Vectastain®, Vector Laboratories, Burlingame, Calif., USA) and Avidin-Biotin-complex (ABC-peroxidase kit; Vectastain®, Vector Laboratories), followed by development with DAB substrate (Vector® DAB SK-4100, Vector Laboratories) for IL-17, and VIP substrate (Vector® VIP SK-4600, Vector Laboratories) for other antibodies.

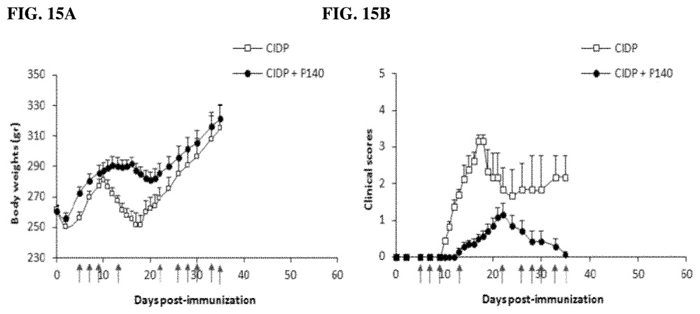

P140 peptide exhibits an effect on the disease severity in CIDP rats and abolishes the chronicity. To examine the effect of P140 peptide on CIDP rats, animals are treated with P140 (100 μg/rat) intraperitoneally at 5, 7, 9, 13 dpi and 3 times per week from 22 dpi until the end of the study. FIG. 15A shows the evolution of weight during the disease course with a maximal weight loss that corresponds to the maximal of clinical scores of the disease. This weight loss is less important in the treated group compared to untreated rats. As shown in FIG. 15B, treatment of P140 not only delayed the onset of the disease and decreased the maximal clinical scores compared to untreated rats but also seems abolish the chronicity of the disease.

### Example 11. Study of the P140 Peptide Effect in a Murine Model of Gougerot-Sjögren Syndrome, the MRL/lpr Mouse (Focus on Salivary Glands)

In this study MRL/lpr female 11-12 week old mice were used with 10 mice per group for statistical analysis. Each mouse received a single injection by retro-orbital, 100 μg of peptide P140 of 100 μl in 9% NaCl. After 5 days, the mouse blood was collected in heparinized tube and salivary glands (GSS) were removed and placed in Eppendorf tubes containing PBS pH 7.4.

The Effects of Peptide P140 have been Studied in Several Systems

Study of cellularity in peripheral blood: 300 μl mouse blood is lysed in of 3 ml DAKO EasyLyse (ref S2364) according to the protocol provided by the Supplier (Procedure B). After two washes in PBS pH 7.4-2% (v/v) fetal calf serum, the cells are taken up in 300 μL of the same buffer. The cells are then counted on Malassez cell in the presence of Turkish Blue to differentiate the leukocytes remaining red blood cells. We infer a number of cells per ml of blood to be compared between different treatment groups to see if the P140 peptide induces a variation in the amount of leukocytes in the blood.

Preparation Organs Cryostat

Salivary glands (SGs) are washed in PBS pH 7.4 and then placed in a cup dedicated to the preparation of cryostat sections. The cup is filled with “OCT” medium (Cell path, ref. 03803126) until the tissue is completely covered. The cup is then immersed in liquid nitrogen and then stored at −80° C. until use.

The tissue was cut by cryostat sections of 5 microns. Sections were left at room temperature overnight (12 hours). The next day the sections were incubated in 100% acetone for 30 minutes. The sections can then be stored at −80° C. for later use. The sections are then rehydrated in PBS pH 7.4, five minutes before immunostaining.

Immunostaining:

The protocol is as follows:

Incubate sections in PBS-2% (w/v) BSA for 30 minutes

Wash Twice 5 minutes with the cuts PBS pH 7.4

Dilute the antibody of interest, typically at 1/200 in PBS-2% BSA and incubated directly on the sections for 2 hours at room temperature (or overnight at 4° C.)

Wash Three times 10 minutes with PBS pH 7.4

Perform nuclear staining with DAPI diluted 1/5000 in PBS for 15 minutes

Wash Three times 10 minutes with PBS pH 7.4

Set sections with paraformaldehyde (PFA) 4% (v/v) for 20 minutes.

Remove excess PFA then mount the cover slip on the slide with the “DAKO mounting medium” and let dry for 2 hours at room temperature, protected from light.

Visualize with microscope.

Marking hematoxylin/eosin:

The number of foci site (FS) is determined for each mouse. A focus is defined as an aggregate of 50 or more cells.

The level of inflammation SG is determined semiquantitatively by a scoring system (0-3 scale): Grade 0: no inflammatory cells; Grade 1: few perivascular inflammatory and periductal Infiltrates (<100 cells); Grade 2: moderate number of perivascular inflammatory and periductal Infiltrates (100-500 cells); Grade 3: extensive inflammation with inflammatory foci broad (>500 cells).

Study of Salivary Glands by Flow Cytometry

Cells of total salivary glands stained with fluorescently were labeled antibodies for 40 min at 4° C., Were Collected data by FACSCalibur.

TABLE 11

| Antibodies | References |
|---|---|
| CD3-FITC | BD-553062 |
| CD4-FITC | BD-557307 |
| CD8-PercP cy5.5 | BD-551162 |
| CD19-PE | BD-553786 |
| CD45-APC | BD-559864 |
| CD45R (B220)-PercP | BD-553093 |
| TCR γσ-APC | eBioscience-17-5711 |
| TCR β-FITC | BD-553170 |

The results of the study of cellularity in the peripheral blood is provided.

The weight of salivary glands Was Measured after-excision. DNase I (1 mg/ml) and collagenase D (50 μg/ml) were used to digest the salivary glands. Total cell counts were evaluated after the digestion.

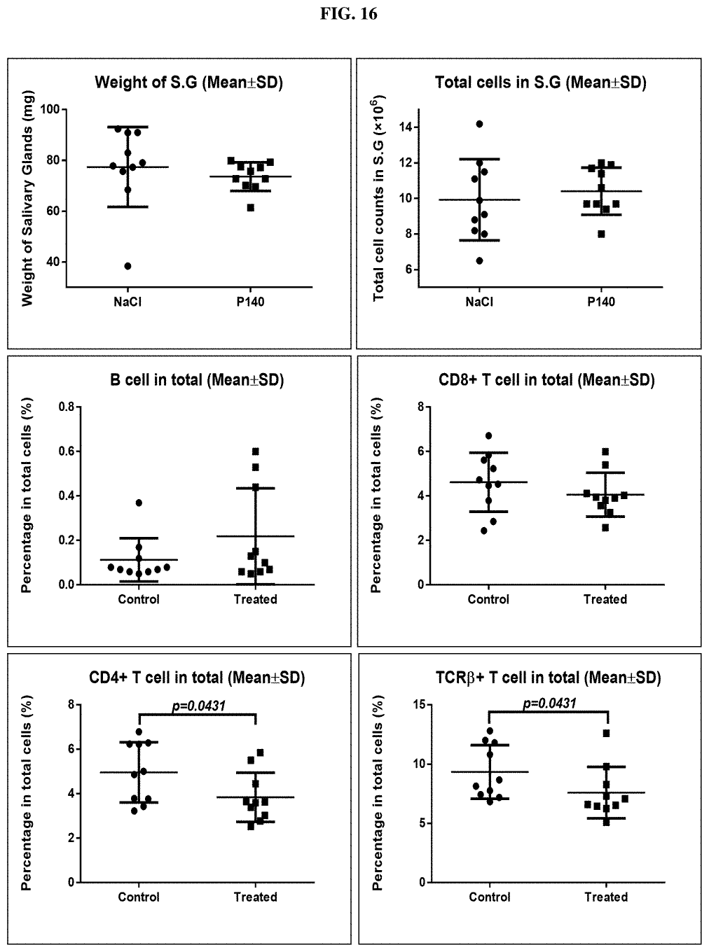

In this experiment the mice were evaluated 5 days post-administration (one single iv injection), P140 peptide had no statistically significant effect on the weight of SGs (FIG. 16).

Study of Salivary Glands by Flow Cytometry

P140 treatment (5 days; one single iv injection) had no apparent effect on the total number of cells present in the SGs Treated of MRL/lpr mice (FIG. 16).

However, when lymphocyte subpopulations were examined, it was detected that the P140 peptide effect was specific to particular lymphocyte subsets. P140 decreased CD4+T cells (but not CD8+T cells) in SGs of MRL/lpr mice (FIG. 16). In preliminary experiments (not shown), we saw that CD4+T cells are the predominant cell subpopulation Infiltrated in SG. These T cells are largely β TCR+T cells. P140 peptide had no statistically significant effect on the total number of B cells.

Study of Salivary Glands in Microscopy

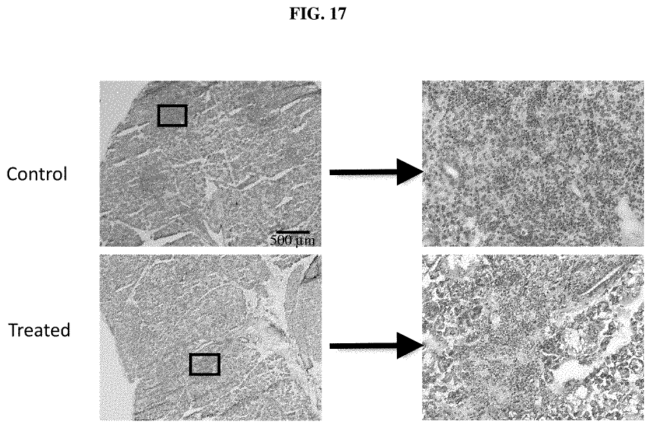

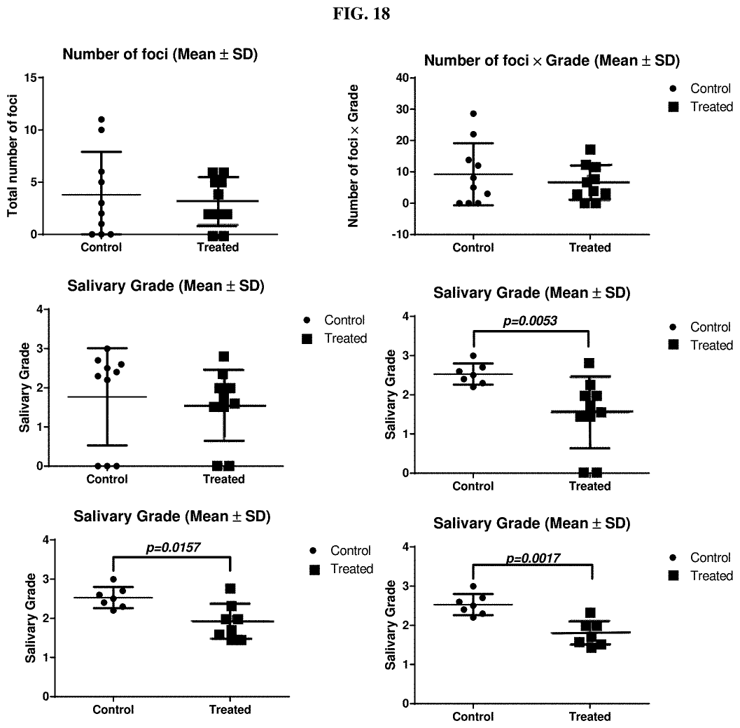

The MRL/lpr mice (10 mice per arm) were injected with peptide P140 (100 μl/mouse iv). Five days after injection the mice were sacrificed and SGs collected as indicated above. The tissue was cut by the cryostat sections of 5 μm. The sections were labeled with hematoxylin/eosin staining is the method most frequently used in tissue histology. The level of inflammation and the number of FS were determined (FIGS. 17 and 18). Representative pictures are from sample control group 4 and group Treated sample (Bar 500 μm).

The results show that as soon as 5 days after one single administration of peptide P140, lymphocytic infiltration in the SGs of MRL/lpr mice was significantly reduced.

### Example 12. Effect of the P140 Peptide in the Murine Model of Rheumatoid Arthritis

Rheumatoid Arthritis (RA) is a chronic inflammatory disease that affects the articulations. The disease evolves by outbreaks of inflammation of varying duration and intensity. In particular, it causes joint swelling in the hands and wrists. Several animal models of RA, usually induced, are available. The following report describes the results obtained in an acute model of RA, namely the model K/B×N mouse. The potential effect of P140 in this mouse has been tested in a “curative” protocol and a “preventive” protocol.

The TCR transgenic mice expressing the KRN and the MHC class II A^{g7} molecule (K/B×N mice) have developed a severe inflammatory arthritis. The administration of serum of these mice to healthy recipient mice causes inflammatory arthritis over a period of about 15 days with a peak ignition around day 7 post-injection.

Two mouse serum administrations from K/B×N were performed (day 0 and day 2). The injection of serum (100 μl/mouse) is performed by intra-peritoneal (ip) injection in mice C57BL/6 (or B6) for 8 weeks (n=10); untreated mouse (n=10).

The P140 peptide (100 μg/100 μl; iv retro-orbital) was administered as follows:

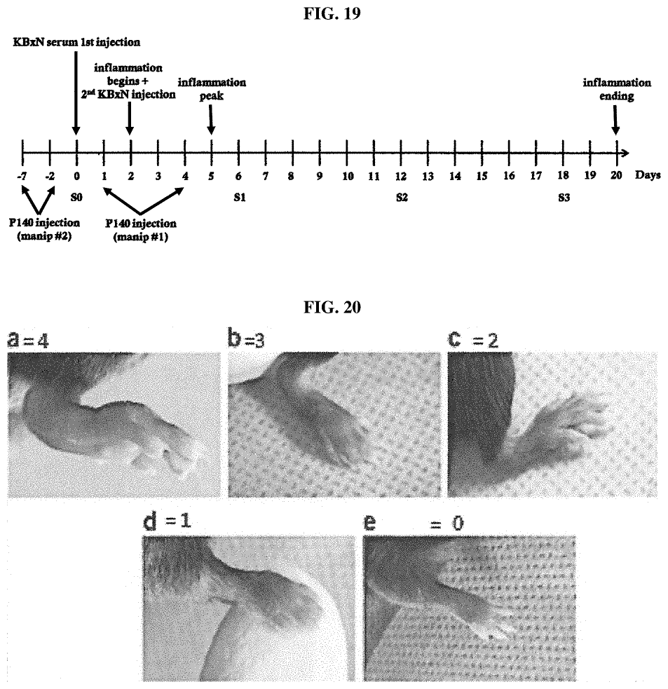

Curative treatment: Injection at day 1 and day 4, to guide the peak of inflammatory disease. Preventive treatment: Injection at day −7 and day −2. Bleeding S0 (at day 0) is followed by bloodletting conducted every six days to dispose of serum. The study ends when inflammation has returned to its basal level, to around day 20 (see FIG. 19).

During the peak of inflammation, every day the animals are evaluated, and swelling score of articulation is established. It is ranged from 0 to 4 and based on a joint observation of the animal. In practice, this score is given for each leg (4 values) and these values are added together to get a general score that ranges from 0 to 16 (FIG. 20).

In this experiment, the induction of the disease has been suboptimal. We did not observe significant increase clinical signs of the disease.

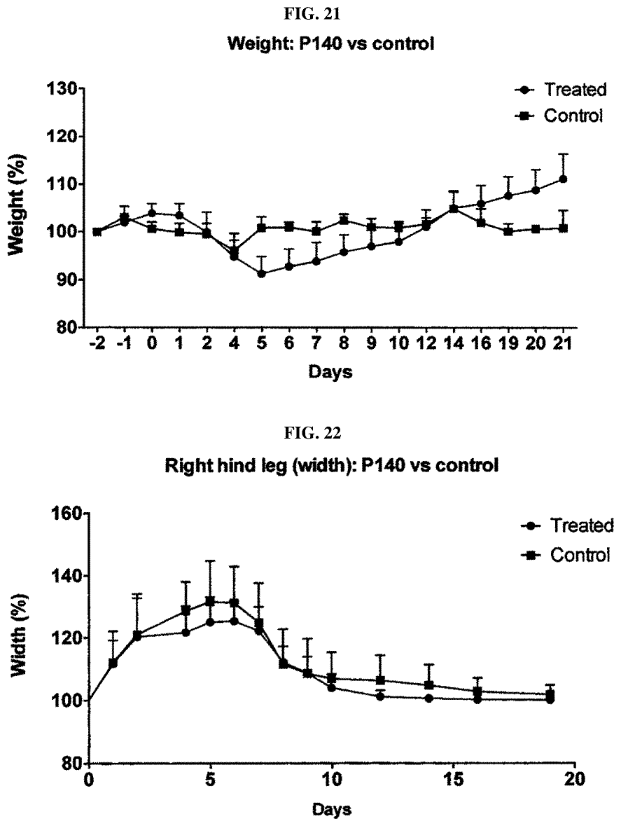

On day 2 (two days after the injection of K/B×N serum, and the day of the 2nd injection serum K/B×N), mice treated P140 NaCl begin to lose weight (15 and 10%). From day 5, the animals begin to regain weight,: we notice a weight gain of 20% for P140 mice treated between day 5 and the end of the study while the mouse controls exceed 5% weight gain. The difference was statistically significant between these two curves (2 way ANOVA) (FIG. 21).

Evolution of the Size of the Legs of the Animals

Right back legs: we see an increase in width of the rear leg from day 0 to day 6, with a maximum between days 5-6 of 30%. From day 6, this increase reverses and we see a return to normal around day 10. The difference was statistically significant between these two curves (2 way ANOVA) (FIG. 22).

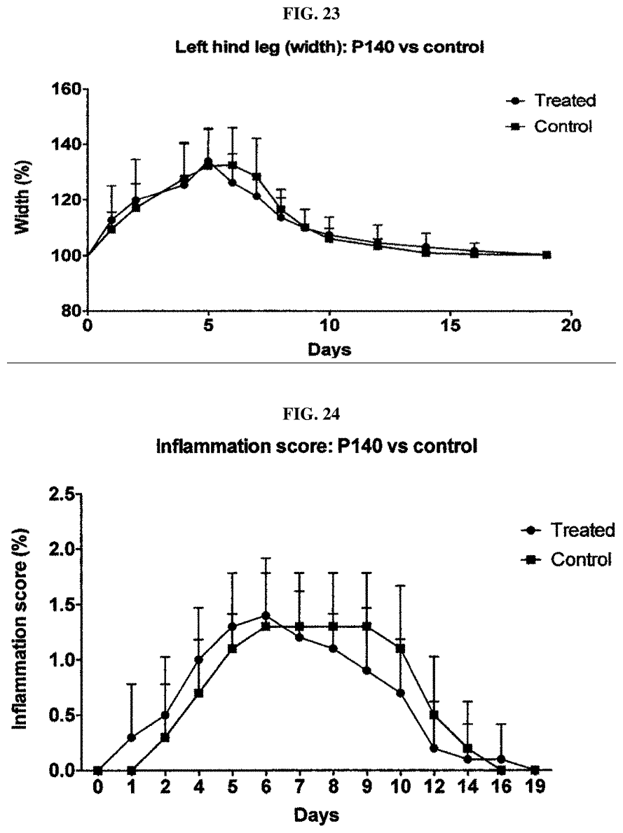

Left back legs: we observe an increase of about 30% of the leg width, with a peak around day 5-6, and then a return to normal gradually, from day 6. The difference was statistically significant between these two curves (2 way ANOVA) (FIG. 23).

Evolution of Inflammation Score

For this experiment, the inflammation scores were calculated independently, for each leg (rear legs left and right). The score exceeds only 1.5 for maximum either for P140 treated mice or mice controls.*For the treated mice the two curves do not show any statistically significant difference in two way ANOVA (FIG. 24).

The results obtained during this preliminary experience have enabled us to identify some important points that will be very useful for the design of the next experiments:

1) inflammation was very moderate (small increase in the size of legs, little weight loss, inflammation of very low scores). The mode of administration serum K/B×N will be changed from 100 μl of serum with 50 μl of vehicle (NaCl) to 100 μl without vehicle.

2) Only the two rear paws of the animal were examined. But ultimately, it was observed that the front legs are most affected by the disease. Next, the four legs of the animal will be taken into account for the measured height joints in foot slides.

3) In the next experiment, an animal's overall inflammation score will be calculated (adding the individual score of the four legs).

Preventive Protocol: Evolution of the Weight of the Animals

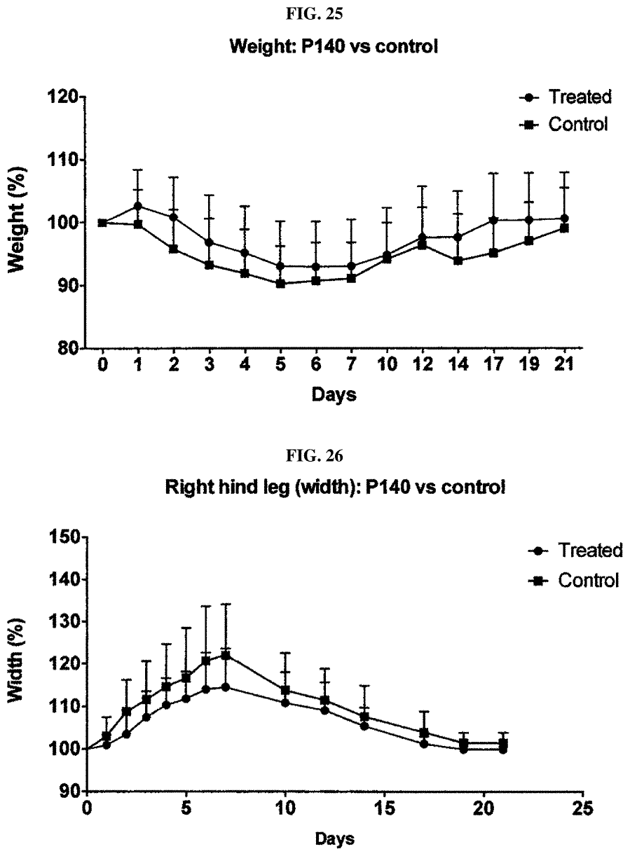

Analysis of the weight of the animals showed a loss of 5% weight-mice treated by the P140 and 10% for controls mouse. This weight loss occurs during the initiation phase (day 1 to day 7). We note a slightly faster return to original weight for mice treated mice compared to controls. However, it is no statistical difference significantly between the two curves (2 way ANOVA) (FIG. 25).

Evolution of the Size of the Legs of the Animals

Right back legs: increasing the size of joints around 12% in treated mice and about 22% in control mice. This increase in size joints occurs between day 0 and day 7, before a return to normal gradually. We note a slight difference in the two curves, but without difference statistically significant (2-way ANOVA) (FIG. 26).

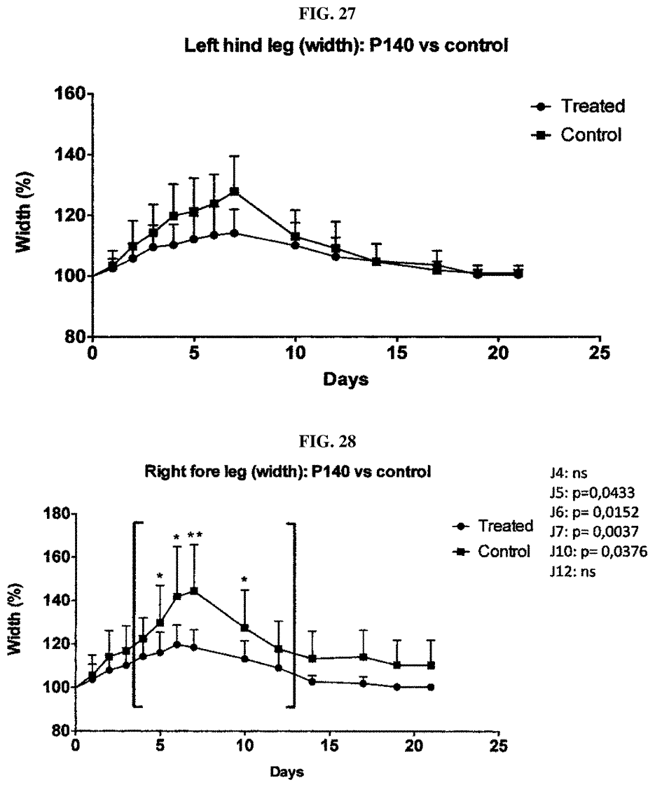

Left hind paws: increase in the size of the joint of about 15% in the treated mice and about 30% among controls mouse. This increase takes place between day 0 and day 7 then a return to normal is observed. We are seeing a lag of two curves, but with no statistical difference significance (2-way ANOVA) (FIG. 27).

Right front legs: as with the rear legs, inflammation occurs between day 0 and day 7 and returns to the normal after day 7 with the treated mice showing moderate swelling in the joints of right front leg (+20%) while the mouse controls undergo an increase of nearly 45%. The difference between the two curves is statistically significant in two way ANOVA (p=0.0069; **) (FIG. 28).

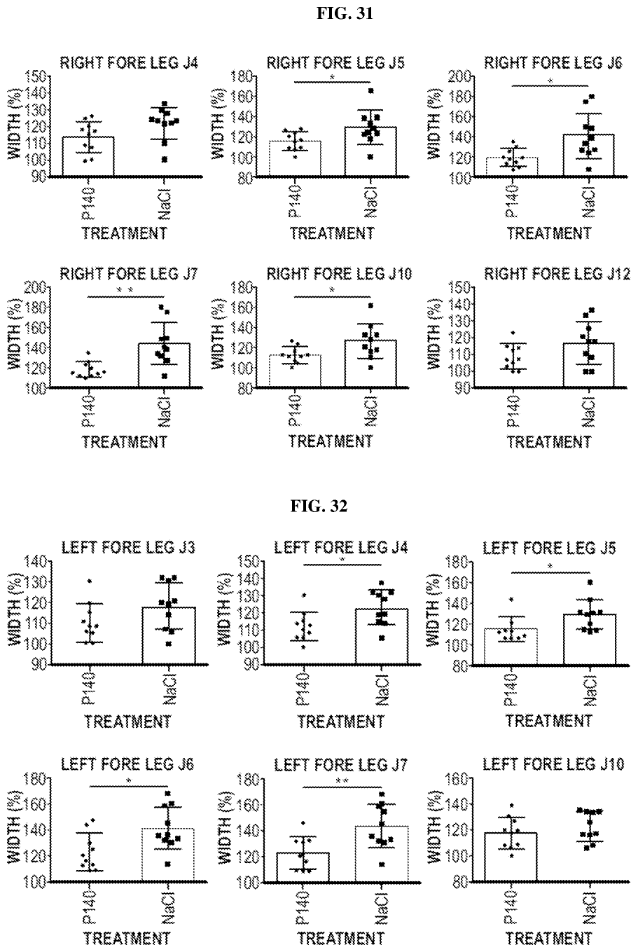

If one compares not the entirety of the curves between them but day by day (unpaired t test) by framing the peak of ignition (between day 4 and day 12; FIG. 28 and FIG. 31), we observe that a maximum of inflammation (day 7), the controls are more affected by the disease than mice treated: p=0.0037; **.

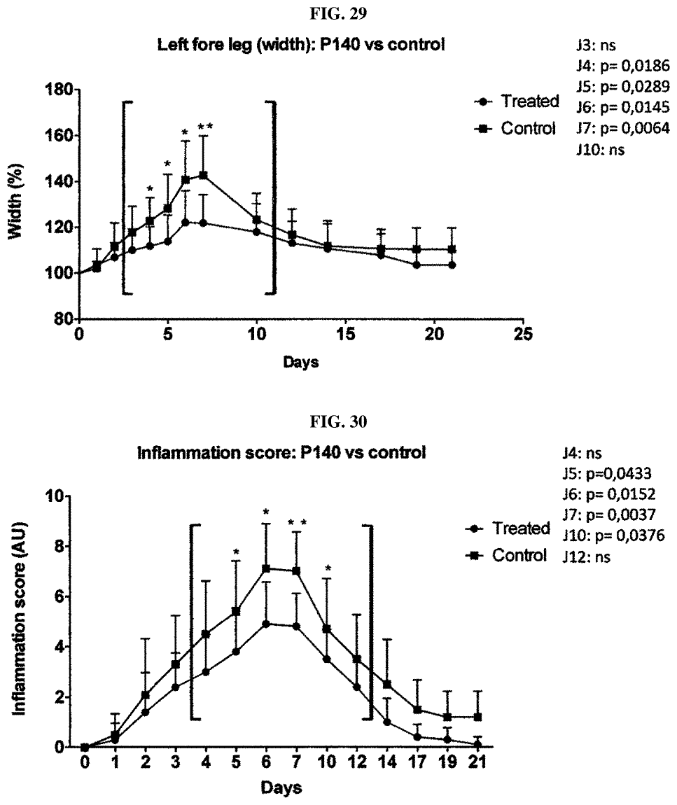

Left front leg: the treated mice showed an increase in the size of their 20% articulation d′− and mouse controls ‘−’ 45%. The difference between the two curves is statistically significant (two way ANOVA—P=0.0397; *) (FIG. 29). We also realized a framework of inflammation from the curves of the growth of the size of the joints of the left front legs. Compared to the previous curve (FIG. 28), it is between about day 3 and day 10.

We note that at peak inflammation (day 7), the controls are more mice affected by clinical signs of disease than mice treated: p=0.0064; **. The representation above (FIG. 29 and FIG. 31) compares daily the growth in the size of the left front legs of mice treated mice compared to controls (Unpaired t test).

Evolution of Inflammation Score

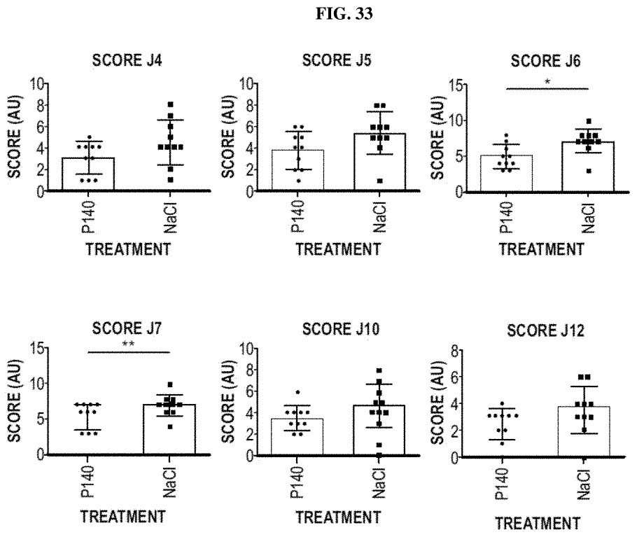

The inflammation scores were calculated independently for each leg (rear legs and front left and right) and then added together to obtain a score of general inflammation for each mouse (FIG. 30). The score for control mice reached a maximum of around day 7 whereas mice treated do not exceed day 5. The framework was realized day 4 to day 12 has on both curves representing revolution of inflammation score (FIG. 30, and FIG. 33). The Two curves are significantly different: p=0.0156; * (Two way ANOVA) (FIG. 30).

In this study, demonstrate an important effect of the P140 peptide in the K/B×N model that mimics RA. All clinical signs (swelling joints, weight loss, and appearance of inflammation score) tend to be attenuated.

In the preventive model and in a statistically significant manner, we find: a loss of less weight of treated mice and a return to normal faster; a lower inflammation in the paws and a limitation of their deformation; their inflammation score decreases sharply when the inflammation is at its maximum.

While preferred embodiments of the present disclosure have been shown and described herein, it will be understood that such embodiments are provided by way of example only. Numerous variations, changes and substitutions will occur to those skilled in the art without departing from the spirit of the present disclosure. Accordingly, it is intended that the appended claims cover all such variations as fall within the spirit and scope of the present disclosure.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the present disclosure described herein. Such equivalents are intended to be encompassed by the following claims. It is understood that the detailed examples and embodiments described herein are given by way of example for illustrative purposes only, and are in no way considered to be limiting to the present disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the present disclosure described herein. Such equivalents are intended to be encompassed by the following claims.

What is claimed is:

1. A method of treating, preventing, or ameliorating at least one symptom of a viral infection or virus-induced immunopathology in a subject in need thereof, the method comprising:
providing a subject in need thereof; and
administering an effective amount of a pharmaceutical composition comprising an effective amount of a peptide having the amino acid sequence as set forth in SEQ ID NO: 4, SEQ ID NO: 5, SEQ ID NO: 6, SEQ ID NO: 7, a salt form thereof, or combination thereof; and at least one pharmaceutically acceptable carrier or excipient, wherein the peptide effectuates the treatment, prevention, or amelioration of at least one symptom of the viral infection or the virus-induced immunopathology.

3. The method of claim 1, wherein the composition comprises an effective amount of a peptide having the amino acid sequence as set forth in SEQ ID NO: 4 or a salt form thereof.

4. The method of claim 1, wherein the composition comprises an effective amount of a peptide having the amino acid sequence as set forth in SEQ ID NO: 5 or a salt form thereof.

5. The method of claim 1, wherein the composition comprises an effective amount of a peptide having the amino acid sequence as set forth in SEQ ID NO: 6 or a salt form thereof.

6. The method of claim 1, wherein the composition comprises an effective amount of a peptide having the amino acid sequence as set forth in SEQ ID NO: 7 or a salt form thereof.

7. The method of claim 1, wherein the virus-induced immunopathology is virus-induced pneumopathy or related to viral pneumonia.

8. The method of claim 1, wherein the viral infection is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, LCMV (lymphocytic choriomeningitis virus), hepatitis B virus, Coxsackie B virus (CBV), Human Immunodeficiency Virus (HIV), Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof.

9. The method of claim 1, wherein the virus-induced immunopathology is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, LCMV (lymphocytic choriomeningitis virus), hepatitis B virus, Coxsackie B virus (CBV), Human Immunodeficiency Virus (HIV), Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof.

10. The method of claim 1, wherein the virus-induced immunopathology is associated with at least one disease selected from: Middle East Respiratory Syndrome (MERS), Severe Acute Respiratory Syndrome (SARS), Coronavirus Disease 2019 (COVID-19), or a combination thereof.

11. The method of claim 1, wherein the method results in a decrease of inflammation (e.g., a decrease in lung inflammation), a decrease in edema (e.g., a decrease of lung edema), a decrease in tissue damage (e.g., a decrease in tissue damage in the lung), ameliorates at least one symptom of the viral infection (e.g., ameliorate at least one system of viral pneumonia), ameliorates at least one symptom of virus-induced immunopathology (e.g., ameliorate at least one system of a virus induced pneumopathy or viral pneumonia), or a combination thereof.

12. The method of claim 1, wherein the method treats, prevents, or ameliorates at least one symptom of COVID-19.

13. The method of claim 1, wherein the method treats, prevents, or ameliorates COVID-19 pathology.

14. A method of modulating the immune response in a subject having a viral infection, the method comprising:
providing a subject in need thereof; and
administering an effective amount of a pharmaceutical composition comprising an effective amount of a peptide having the amino acid sequence as set forth in SEQ ID NO: 4, SEQ ID NO: 5, SEQ ID NO: 6, SEQ ID NO: 7, a salt form thereof, or combination thereof; and at least one pharmaceutically acceptable carrier or excipient, wherein the peptide effectuates the treatment, prevention, or amelioration of at least one symptom of virus-induced immunopathology.

15. The method of claim 14, wherein the composition comprises an effective amount of a peptide having the amino acid sequence as set forth in SEQ ID NO: 4 or a salt form thereof.

16. The method of claim 14, wherein the composition comprises an effective amount of a peptide having the amino acid sequence as set forth in SEQ ID NO: 5 or a salt form thereof.

17. The method of claim 14, wherein the composition comprises an effective amount of a peptide having the amino acid sequence as set forth in SEQ ID NO: 6 or a salt form thereof.

18. The method of claim 14, wherein the composition comprises an effective amount of a peptide having the amino acid sequence as set forth in SEQ ID NO: 7 or a salt form thereof.

19. The method of claim 14, wherein the virus-induced immunopathology is virus-induced pneumopathy or related to viral pneumonia.

20. The method of claim 14, wherein the viral infection is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, LCMV (lymphocytic choriomeningitis virus), hepatitis B virus, Coxsackie B virus (CBV), Human Immunodeficiency Virus (HIV), Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof.

21. The method of claim 14, wherein the virus-induced immunopathology is caused by at least one virus selected from: Coronavirus, Severe Acute Respiratory Syndrome Coronavirus 2 (SARS-CoV-2), Middle East Respiratory Syndrome Coronavirus (MERS-CoV), Severe Acute Respiratory Syndrome Coronavirus (SARS-CoV), Respiratory Syncytial Virus (RSV), Rhinovirus, Influenza A, Influenza B, Influenza C, Human metapneumovirus, LCMV (lymphocytic choriomeningitis virus), hepatitis B virus, Coxsackie B virus (CBV), Human Immunodeficiency Virus (HIV), Parainfluenza virus type 1, Parainfluenza virus type 2, Parainfluenza virus type 3, Parainfluenza virus type 4, Adenovirus, Enterovirus, Varicella-zoster virus, Hantavirus, Epstein-Barr virus (EBV), Herpes Simplex Virus, Cytomegalovirus (CMV), or a combination thereof.

22. The method of claim 14, wherein the virus-induced immunopathology is associated with at least one disease selected from: Middle East Respiratory Syndrome (MERS), Severe Acute Respiratory Syndrome (SARS), Coronavirus Disease 2019 (COVID-19), or a combination thereof.

23. The method of claim 14, wherein the method results in a decrease of inflammation (e.g., a decrease in lung inflammation), a decrease in edema (e.g., a decrease of lung edema), a decrease in tissue damage (e.g., a decrease in tissue damage in the lung), ameliorates at least one symptom of the viral infection (e.g., ameliorate at least one system of viral pneumonia), ameliorates at least one symptom of virus-induced immunopathology (e.g., ameliorate at least one system of a virus induced pneumopathy or viral pneumonia), or a combination thereof.

24. The method of claim 14, wherein the method treats, prevents, or ameliorates at least one symptom of COVID-19.

25. The method of claim 14, wherein the method treats, prevents, or ameliorates COVID-19 pathology.


SEQUENCE LISTING (8 sequences)

SEQ ID NO: 1 - protein, 21 aa, Artificial sequence
  derived from U1 snRNP 70 kDa
RIHMVYSKRSGKPRGYAFIEY

SEQ ID NO: 2 - protein, 20 aa, Artificial sequence
  derived from U1 snRNP 70 kDa
IHMVYSKRSGKPRGYAFIEY

SEQ ID NO: 3 - protein, 437 aa, Homo sapiens
MTQFLPPNLLALFAPRDPIPYLPPLEKLPHEKHHNQPYCGIAPYIREFEDPRDAPPPTRAETREERMERKRREKIERRQQEVETELKMWDPHNDPNAQGDAFKTLFVARVNYDTTESKLRREFEVYGPIKRIHMVYSKRSGKPRGYAFIEYEHERDMHSAYKHADGKKIDGRRVLVDVERGRTVKGWRPRRLGGGLGGTRRGGADVNIRHSGRDDTSRYDERPGPSPLPHRDRDRDRERERRERSRERDKERERRRSRSRDRRRRSRSRDKEERRRSRERSKDKDRDRKRRSSRSRERARRERERKEELRGGGGDMAEPSEAGDAPPDDGPPGELGPDGPDGPEEKGRDRDRERRRSHRSERERRRDRDRDRDRDREHKRGERGSERGRDEARGGGGGQDNGLEGLGNDSRDMYMESEGGDGYLAPENGYLMEAAPE

SEQ ID NO: 4 - protein, 21 aa, Artificial sequence
  derived from U1 snRNP 70 kDa
  MOD_RES (10)..(10): PHOSPHORYLATION
RIHMVYSKRSGKPRGYAFIEY

SEQ ID NO: 5 - protein, 20 aa, Artificial sequence
  derived from U1 snRNP 70 kDa
  MOD_RES (9)..(9): PHOSPHORYLATION
IHMVYSKRSGKPRGYAFIEY

SEQ ID NO: 6 - protein, 21 aa, Artificial sequence
  derived from U1 snRNP 70 kDa
  MOD_RES (4)..(4): OXIDATION
  MOD_RES (10)..(10): PHOSPHORYLATION
RIHMVYSKRSGKPRGYAFIEY

SEQ ID NO: 7 - protein, 20 aa, Artificial sequence
  derived from U1 snRNP 70 kDa
  MOD_RES (3)..(3): OXIDATION
  MOD_RES (9)..(9): PHOSPHORYLATION
IHMVYSKRSGKPRGYAFIEY

SEQ ID NO: 8 - DNA, 66 bp, Artificial Sequence
  derived from U1 snRNP 70 kDa
  misc_feature (3)..(3): n is a, c, g, or t
  misc_feature (15)..(15): n is a, c, g, or t
  misc_feature (21)..(21): n is a, c, g, or t
  misc_feature (27)..(27): n is a, c, g, or t
  misc_feature (30)..(30): n is a, c, g, or t
  misc_feature (33)..(33): n is a, c, g, or t
  misc_feature (39)..(39): n is a, c, g, or t
  misc_feature (42)..(42): n is a, c, g, or t
  misc_feature (45)..(45): n is a, c, g, or t
  misc_feature (51)..(51): n is a, c, g, or t
mgnathcayatggtntaywsnaarmgnwsnggnaarccnmgnggntaygcnttyathgartaytrr